(19) United States
(12) Patent Application Publication
(10) Pub. No.: US 2025/0326794 A1
(43) Pub. Date: Oct. 23, 2025

(54) ANTI-CANCER LEUCINE-RICH PEPTIDES AND USES THEREOF

(71) Applicant: KING'S COLLEGE LONDON, London (GB)

(72) Inventors: Martin Bernhard Ulmschneider, London (GB); Charles Huang Chen, London (GB)

(73) Assignee: KING'S COLLEGE LONDON, London (GB)

(21) Appl. No.: 17/767,859

(22) PCT Filed: Oct. 9, 2020
(86) PCT No.: PCT/GB2020/052510

§ 371 Date: Apr. 8, 2022

(30) Foreign Application Priority Data
Oct. 10, 2019 (GB) 1914682.8

(51) Int. Cl. C07K 7/08; A61K 38/00; A61P 35/00

(52) U.S. Cl. CPC C07K 7/08; A61P 35/00; A61K 38/00

(57) ABSTRACT

The invention relates to a pharmaceutically acceptable composition for use in the treatment of cancer, the composition comprising one or more peptides having a sequence comprising the motif GLLxLLxLLLxAAG, wherein x is independently selected from arginine (R), histidine (H), lysine (K), aspartic acid (D) or glutamic acid (E), and one or more pharmaceutically acceptable excipients. The invention also relates to the peptides of the pharmaceutically acceptable composition, a kit comprising the pharmaceutically acceptable composition, nucleotides encoding the peptides and vectors expressing the peptides

### FIELD OF THE INVENTION

The invention relates to a family of anti-cancer peptides (ACPs) which can be used in the treatment of cancer.

### BACKGROUND TO THE INVENTION

Tumours are heterogeneous at the cellular level, consisting of a range of different subtypes of cancer cells. Among these subtypes, cancer stem cells (CSCs) are increasingly recognised as a major difficulty in traditional pharmaceutical treatment using current anti-cancer drugs. Breast cancer is the second most common cancer around the world, and mostly occurs in women. Several studies have shown that breast cancer stem cells might develop resistance to conventional anti-cancer drugs to survive, self-renew, differentiate and relapse.¹⁻⁶ CSCs readily evolve resistance to anticancer drugs and the chemotherapeutic treatment of solid tumours typically results in a significant increase in the share of drug-resistant CSCs in the patient. This can lead to relapse and the formation of metastases. Furthermore, it is possible that breast tumours can be different within the same patient and conventional anticancer drugs may fail.⁷⁻⁹ Treatment with higher doses is difficult as commonly used anticancer drugs, such as doxorubicin, have a generally high toxicity towards healthy tissues, resulting in acute damage to organs such as the liver, kidneys, and heart.¹⁰⁻¹² Therefore, there is an urgent, unmet need to develop new anti-cancer drugs that have improved selectivity towards cancer cells, leaving healthy tissues unharmed at doses that are sufficient to kill all bulk cancer and CSCs in a solid tumour.

### SUMMARY OF THE INVENTION

In a first aspect of the invention, there is provided a pharmaceutically acceptable composition for use in the treatment of cancer, the composition comprising one or more peptides having a sequence comprising the motif GLLxLLxLLLxAAG, wherein each x is independently selected from arginine (R), histidine (H), lysine (K), aspartic acid (D) or glutamic acid (E), and one or more pharmaceutically acceptable excipients.

The inventors have surprisingly found that a family of peptides conforming to the claimed formula have improved selectivity towards cancer cells, leaving healthy tissues unharmed at doses that are sufficient to kill all bulk cancer and CSCs in a solid tumour. Unlike many conventional anticancer drugs, the pore-forming membrane-active peptides developed here target and disrupt the plasma membrane to kill cancer cells. This removes the complication of having to transport the drug into the cytoplasm and as such, the peptides have improved tumour penetration in comparison to traditional chemotherapy agents. The presently claimed peptides act by selectively targeting the plasma membranes of cancer cells and forming pores therein, thus killing the cells by short-circuiting their electrochemical gradient. Without wishing to be bound by theory, it is thought that the peptides directly target the lipid composition and chemical microenvironment of the cancer cell membrane. Consequently, the peptides are far less likely to induce resistance (in a similar way that it is difficult for cells to develop resistance to detergents) as it is difficult for the tumour cells to modify their lipid composition.¹³⁻¹⁵

Several of the disclosed peptides have nano-molar activity against bulk cancer and CSCs, comparable to current approved anti-cancer drugs such as salinomycin. Furthermore, in one of the best current in vitro breast cancer models, the mammosphere model, which mimics a real solid tumour by growing cells into a spherical clump, several of the peptides disclosed herein exhibit superior activity against cancer cells, while retaining reduced toxicity towards normal, healthy cells.

The peptides work in both the L and D amino acid forms (the latter being a major advantage for in vivo stability against protease degradation) to selectively eliminate two-dimensionally grown cancer cells, as well as three-dimensional (spheroid) cancer cell cultures at very low micromolar, and in some cases, nanomolar concentrations. 3 to >200-fold higher concentrations are required to harm non-cancerous human breast and kidney cells.

The peptides are inexpensive and straightforward to synthesize, are easy to modify and high-throughput screen, and offer a chemical and structural repertoire to target cancer cells specifically.

The presently claimed peptides are de novo designed, and have no known natural analogues, as confirmed by comparison with extant peptide databases. Short flexible peptides of this type will have low immunogenicity and are thus suitable for pharmaceutical applications.

As herein described the term “peptide” refers to any peptide comprising amino acids joined to each other by peptide bonds or modified peptide bonds, i.e., peptide isosteres. The peptide generally will contain naturally occurring amino acids, but may include amino acid sequences modified either by natural processes, such as post-translational processing, or by chemical modification techniques, which are well known in the art. Such modifications are well described in basic texts. Modifications can occur anywhere in a peptide, including the peptide backbone, the amino acid side-chains and the amino or carboxyl termini. It will be appreciated that the same type of modification may be present in the same or varying degrees at several sites in a given peptide. Also, a given peptide may contain many types of modifications.

Preferably, the peptides are isolated peptides. The term “isolated” means that the peptide is removed from its original environment. For example, a peptide present in a living animal is not isolated, but the same peptide, or a fragment of such a peptide, separated from some or all of the coexisting materials in the natural system, is isolated. Such peptides could be part of a vector and/or peptides could be part of a composition, and still be isolated in that such vector or composition is not part of its natural environment.

The pharmaceutical composition comprising the peptides may be for human or animal usage in human and veterinary medicine and will typically comprise one or more suitable excipients. Acceptable excipients for therapeutic use are well known in the pharmaceutical art, and are described, for example, in Remington's Pharmaceutical Sciences, Mack Publishing Co. (A. R. Gennaro edit. 1985). The choice of pharmaceutical excipient can be selected with regard to the intended route of administration and standard pharmaceutical practice. The pharmaceutical compositions may comprise as, or in addition to, the excipient, any suitable binder, lubricant, suspending agent, coating agent or solubilising agent.

Preservatives, stabilizers and dyes may be provided in the pharmaceutical composition.

Examples of preservatives include sodium benzoate, sorbic acid and esters of p-hydroxybenzoic acid. Antioxidants and suspending agents may be also used.

The pharmaceutical composition may also comprise tolerance-promoting adjuvants and/or tolerance promoting cells. Tolerance promoting adjuvants include IL-10, recombinant cholera toxin B-subunit (rCTB), ligands for Toll-like receptor 2, as well as biologics and monoclonal antibodies that modulate immune responses, such as anti-CD3 and co-stimulation blockers, which may be co-administered with the peptide. Tolerance promoting cells include immature dendritic cells and dendritic cells treated with vitamin D3, (1alpha,25-dihydroxy vitamin D3) or its analogues.

When cancer is “treated”, this means that one or more clinical manifestations of cancer are ameliorated. It does not mean that the symptoms of cancer are completely remedied so that they are no longer present in the patient, although in some methods, this may be the case. “Treatment” results in one or more of the symptoms of cancer being less severe than before treatment. For example, a tumour may be reduced in size or eradicated entirely.

A second aspect of the invention relates to a pharmaceutically acceptable composition for use in the manufacture of a medicament for the treatment of cancer, the composition comprising one or more peptides having a sequence comprising the motif GLLxLLxLLLxAAG, wherein each x is independently selected from arginine (R), histidine (H), lysine (K), aspartic acid (D) or glutamic acid (E), and one or more pharmaceutically acceptable excipients.

In one embodiment, the peptide may comprise a sequence be selected from any one of SEQ ID NO: 1 to 36 or mixtures thereof. In a further embodiment, the peptide may consist of the sequence of any one of SEQ ID NO: 1 to 36.

In one embodiment, the pharmaceutically acceptable composition comprises a peptide having a sequence comprising the motif GLLxLLELLLxAAG, wherein x is selected from arginine (R), histidine (H), lysine (K), aspartic acid (D) or glutamic acid (E) and mixtures thereof. The inventors have surprisingly found that peptides with this sequence have a better selectivity for cancer cells.

In one embodiment, the pharmaceutically acceptable composition comprises a peptide having a sequence comprising the motif GLLxLLxLLLxAAG, wherein x is selected from arginine (R), histidine (H), lysine (K), aspartic acid (D) or glutamic acid (E) and mixtures thereof, but wherein the sequence does not comprise SEQ ID NO: 29 or SEQ ID NO: 33.

In one embodiment, the pharmaceutically acceptable composition comprises a sequence selected from SEQ ID NO: 2, SEQ ID NO: 4, SEQ ID NO: 6, SEQ ID NO: 14, SEQ ID NO: 25 or SEQ ID NO: 26 and mixtures thereof. More preferably, the pharmaceutically acceptable composition comprises a sequence selected from SEQ ID NO: 2, SEQ ID NO: 4, SEQ ID NO: 14, SEQ ID NO: 25 or SEQ ID NO: 26 and mixtures thereof. Even more preferably, the pharmaceutically acceptable composition comprises a sequence selected from SEQ ID NO: 25 and/or SEQ ID NO: 26. The inventors have found that these sequences have a particularly selective for cancer cells.

The pharmaceutically acceptable composition of the present invention may be used to treat any type of cancer such as skin cancer, lung cancer, breast cancer, prostate cancer, colorectal cancer, bladder cancer, lymphomas, kidney cancer, pancreatic cancer or endometrial cancer. However, in a particular embodiment of the invention, the cancer is breast cancer.

In one embodiment, the pharmaceutically acceptable composition comprises a peptide, which further comprises a tryptophan residue (W) at the C-terminus of the motif. This helps with accurate concentration measurements and precise dosing.

The N- and C-termini of the peptide sequence or motif may be any termini known to one skilled in the art and may include NH₂, NH₃⁺, COOH and COO⁻ for example.

In one embodiment, the pharmaceutically acceptable composition comprises a peptide wherein the peptide sequence consists of the motif GLLxLLxLLLxAAG.

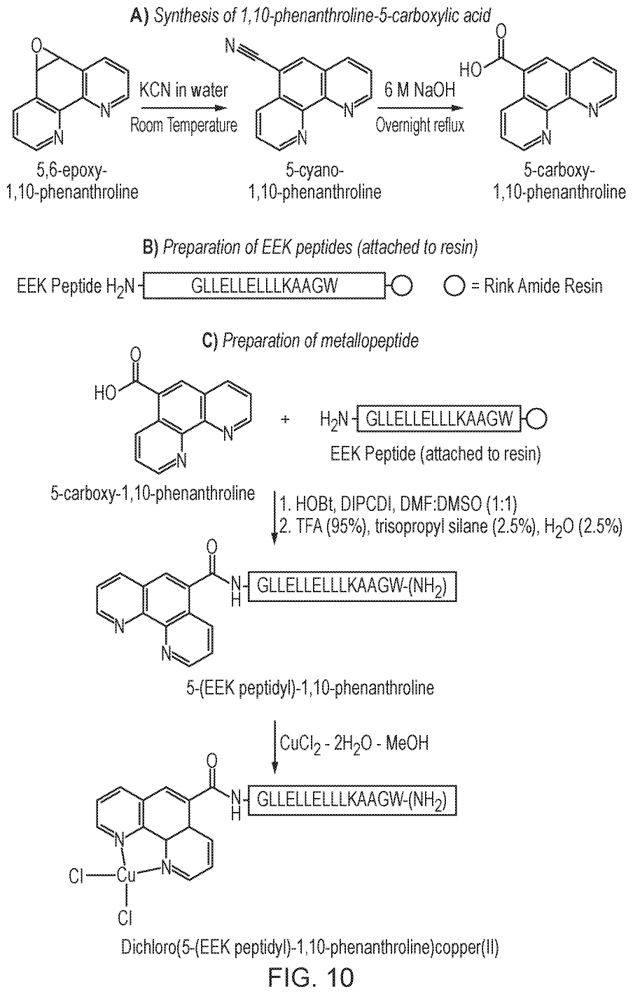

In one embodiment of the present invention, the composition is for use in combination with a chemotherapy agent. The inventors have found that due to the pore forming properties of the presently claimed peptides, this grants easier access to the target cancer cells for standard chemotherapeutic agents. The chemotherapeutic agent may be selected from cyclophosphamide, methotrexate, 5-fluorouracil, vinorelbine, doxorubicin, docetaxel, bleomycin, vinblastine, dacarbazine, mustine, vincristine, procarbazine, prednisolone, etoposide, cisplatin, epirubicin, methotrexate, capecitabine, vinorelbine, folinic acid, oxaliplatin and mixtures thereof. Preferably the chemotherapeutic agent is doxorubicin. One example of a means to conjugate the present peptides to a chemotherapeutic agent is provided in FIG. 10.

There may be different composition/formulation requirements for the pharmaceutical composition dependent on the chosen delivery system. By way of example, the pharmaceutical composition of the present invention may be formulated to be delivered parenterally in which the composition is formulated in an injectable form, for delivery, by, for example, an intravenous, intradermal, intramuscular, subcutaneous or intraperitoneal route. For parenteral administration, the compositions may be best used in the form of a sterile aqueous solution which may contain other substances, for example enough salts or monosaccharides to make the solution isotonic with blood. Intradermal administration routes include any dermal-access means, for example, using microneedle-based injection and infusion systems (or other means to accurately target the intradermal space), needleless or needle-free ballistic injection of fluids or powders into the intradermal space, Mantoux-type intradermal injection, enhanced iontophoresis through microdevices, and direct deposition of fluid, solids, or other dosing forms into the skin, including the use of patches to deposit the composition onto the skin. The composition may also be formulated to be administered by oral or topical routes, including nasally, orally or epicutaneously. Preferably the composition is formulated to be delivered by an intravenous route.

The amount or dose of the disclosed anticancer peptides that is administered should be sufficient to effectively target cancer cells in vivo. The dose will be determined by the efficacy of the particular formulation and the location of the tumour in the subject, as well as the body weight of the subject to be treated.

The dose of the disclosed anticancer peptides will also be determined by the existence, nature, and extent of any adverse side effects that might accompany the administration of a particular formulation. Typically, a physician will decide the dosage of the peptides with which to treat each individual subject, taking into consideration a variety of factors, such as age, body weight, general health, diet, sex, compound/formulation to be administered, route of administration, and the severity of the condition being treated. The appropriate dosage can be determined by one skilled in the art. By way of non-limiting example, the total dose of the anticancer peptides of the present invention can be about 0.001 to about 1000 mg/kg body weight of the subject being treated, from about 0.01 to about 100 mg/kg body weight, from about 0.1 mg/kg to about 10 mg/kg, and from about 0.5 mg to about 5 mg/kg body weight. In another embodiment, the total dose of the peptides can be at a concentration from about 1 nM to about 10,000 nM, preferably from about 10 nM to about 5,000 nM, more preferably from about 100 nM to about 500 nM.

In a preferred embodiment, the composition comprising the peptide of the present invention is administered at least once per month, preferably once every 1 to 4 weeks for four administrations.

The peptides can be present in either the D or the L form. In one embodiment, the pharmaceutically acceptable composition comprises a peptide in the L form. It has been surprisingly found by the inventors that the peptides presented here are more selective for cancer cells when in the L form.

In one embodiment, the pharmaceutically acceptable composition comprises a peptide which forms an alpha helical assembly. Preferably the peptide forms a pore in a cancer cell membrane. It is believed that the peptides directly target the lipid composition and chemical microenvironment of the cancer cell membrane and form pores therein that kill the cancer cells by short-circuiting their electrochemical gradient.

A third aspect of the invention relates to a method of treatment of cancer in which the pharmaceutically acceptable composition of the invention is administered to a patient with cancer. In one embodiment the cancer is breast cancer.

A fourth aspect of the invention relates to a peptide having a sequence comprising the motif GLLxLLELLLxAAG, wherein each x is independently selected from arginine (R), histidine (H), lysine (K), aspartic acid (D) or glutamic acid (E).

A fifth aspect of the invention relates to a peptide having a sequence comprising the motif GLLxLLxLLLxAAG, wherein x is wherein each x is independently selected from arginine (R), histidine (H), lysine (K), aspartic acid (D) or glutamic acid (E) and wherein the sequence does not comprise SEQ ID NO: 29 or SEQ ID NO: 33.

A sixth aspect of the invention relates to a kit for treating cancer comprising the pharmaceutically acceptable composition of the invention. In a preferred embodiment, the kit is for treating breast cancer. The kit may further comprise a chemotherapeutic agent.

A seventh aspect of the invention relates to a nucleotide sequence encoding a peptide comprising the sequence of any one of SEQ ID NO: 1 to 36.

An eight aspect of the invention relates to a vector expressing a peptide comprising the sequence of any one of SEQ ID NO: 1 to 36 and mixtures thereof.

The vector may be any appropriate vector for expressing the peptides of the present invention, including viral and non-viral vectors. Viral vectors include a parvovirus, an adenovirus, a retrovirus, a lentivirus or a herpes simplex virus. The parvovirus may be an adenovirus-associated virus (AAV). The vector is preferably a recombinant adeno-associated viral (rAAV) vector or a lentiviral vector. More preferably, the vector is a rAAV vector.

A vector according to the invention may be a gene delivery vector. Such a gene delivery vector may be a viral gene delivery vector or a non-viral gene delivery vector.

Accordingly, the present invention provides gene delivery vectors based on animal parvoviruses, in particular dependoviruses such as infectious human or simian AAV, and the components thereof (e.g., an animal parvovirus genome) for use as vectors for introduction and/or expression of the peptides of the present invention in a mammalian cell. The term “parvoviral” as used herein thus encompasses dependoviruses such as any type of AAV.

A skilled person will appreciate that all aspects of the invention, whether they relate to, for example, the pharmaceutically acceptable composition, peptide, its use, or a method of treatment, are equally applicable to all other aspects of the invention. In particular, aspects of the pharmaceutically acceptable composition for example, may have been described in greater detail than in other aspects of the invention, for example, the peptide per se. However, the skilled person will appreciate where more detailed information has been given for a particular aspect of the invention, this information is generally equally applicable to other aspects of the invention.

### DETAILED DESCRIPTION OF THE INVENTION

The invention will now be described in detail by way of example only with reference to the figures in which:

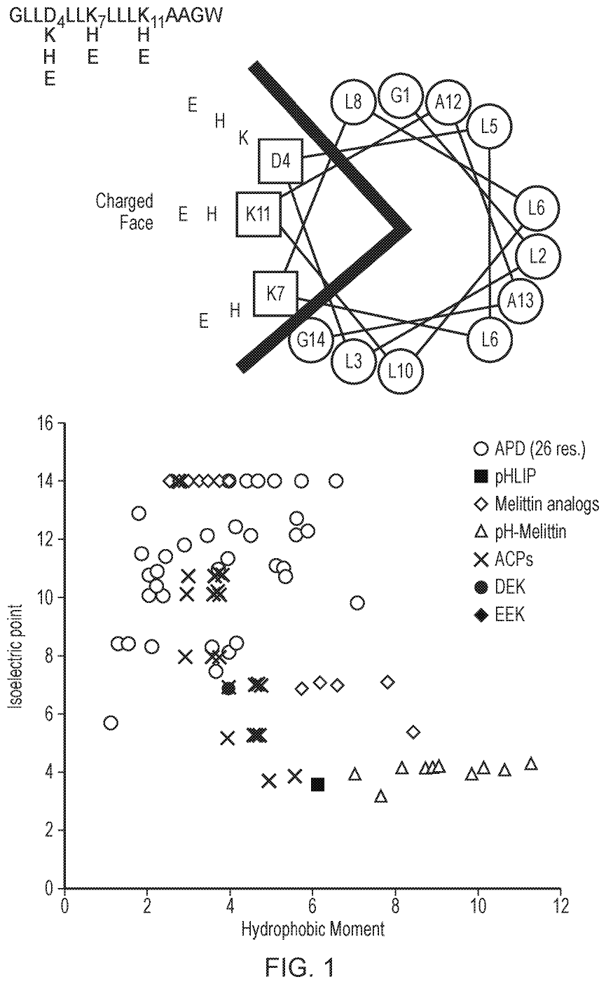

FIG. 1 shows the design of a combinatorial leucine-rich peptide library and comparison with other pore-forming and cancer targeting membrane active peptides. A) Combinatorial peptide library sequences are shown together with their projection onto a helical wheel, which is the presumed membrane-active conformation. B) Comparison of the isoelectric point and hydrophobicity of the library peptides to other pore forming and cancer-targeting membrane-active peptides. Peptides that contain 26 amino acids in the antimicrobial peptide database (APD), melittin and its analogs (gain-of-function and loss-of-function analogs), pH-dependent melittin, and the cancer targeting pH-low insertion peptide (pHLIP).

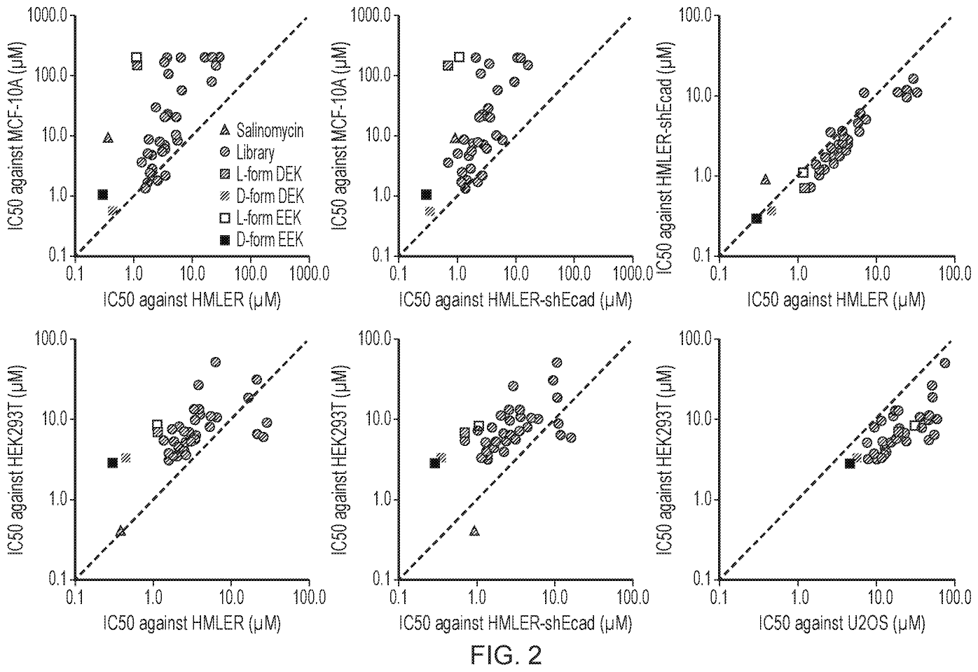

FIG. 2 shows the results of an in vitro cytotoxicity screen of the library of the presently identified sequences, consisting of 36 combinatorial peptides (SEQ ID NO: 1 to 36) against different human cell lines, derived from both cancerous and healthy human tissues. Also shown are in vitro cytotoxicity screening results for selected D-form peptides, as well as the clinically used anticancer drugs salinomycin and doxorubicin. Cytotoxicity was evaluated for different human cell lines and is quantified using the half maximal inhibitory concentration (IC₅₀) for: A) HMLER versus MCF-10A, B) HMLER-shEcad versus MCF-10A, C) HMLER versus HMLER-shEcad, D) HMLER versus HEK293T, E) HMLER-shEcad versus HEK293T, and F) U2OS versus HEK293T.

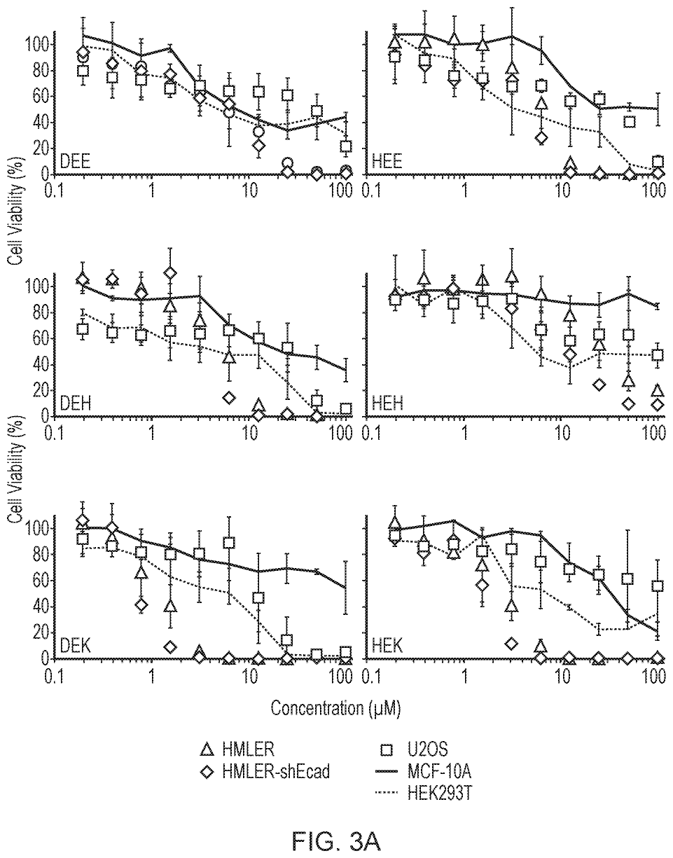

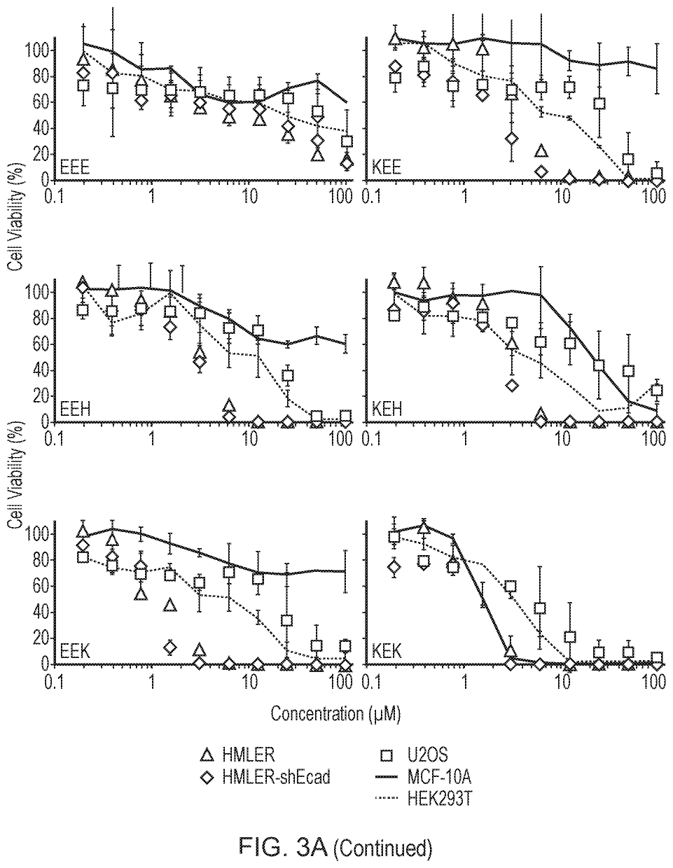

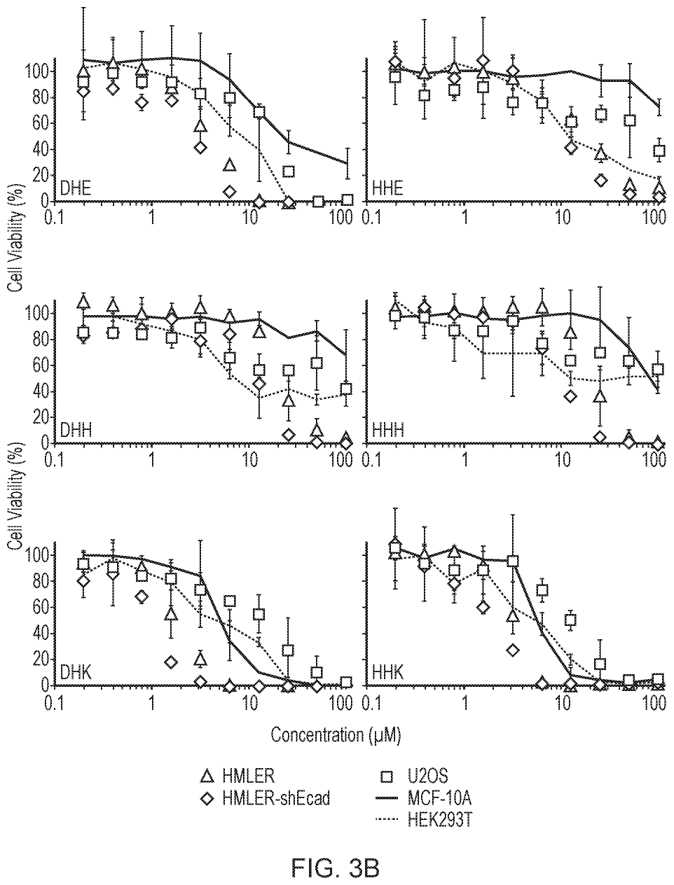

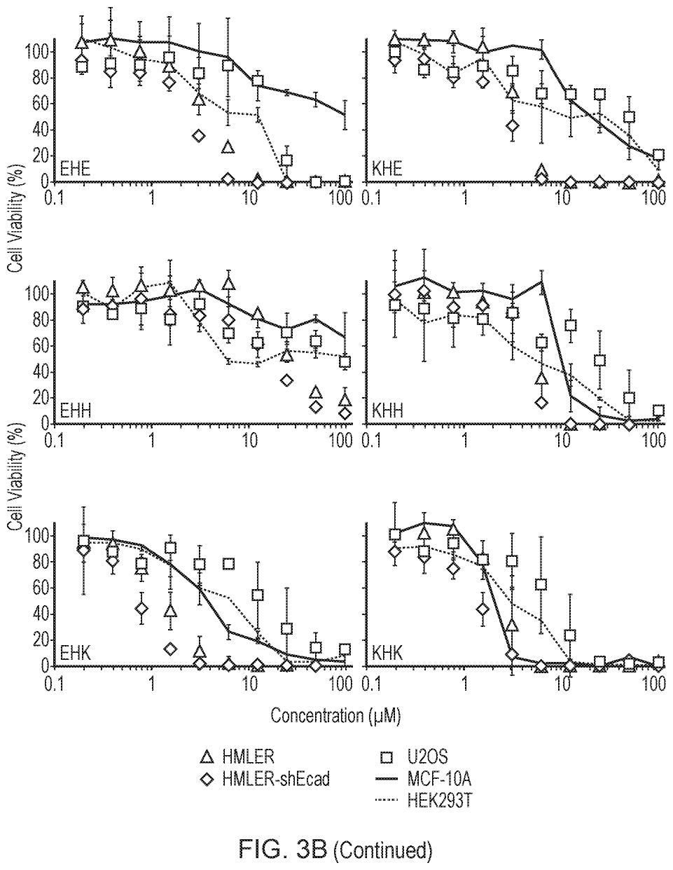

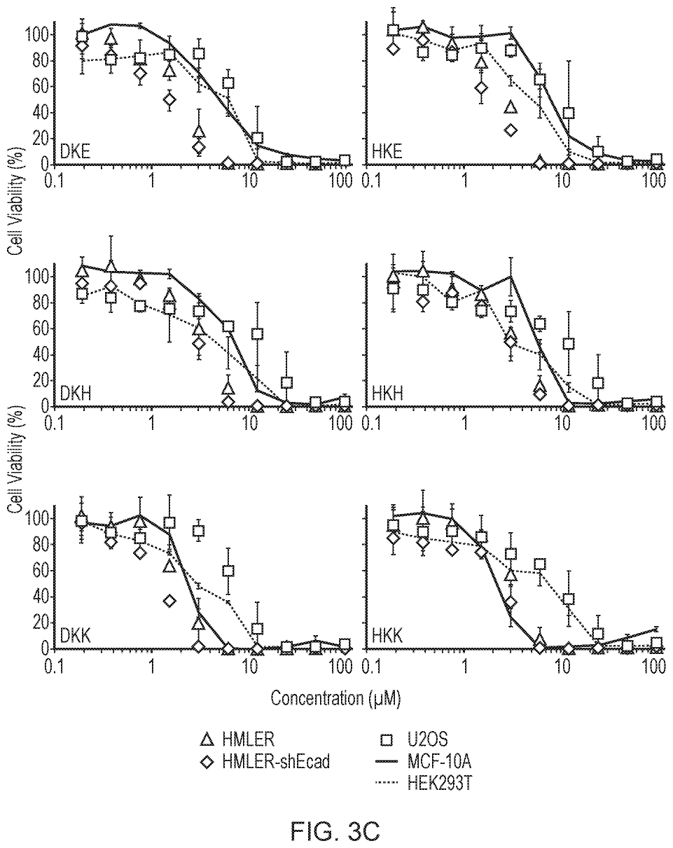

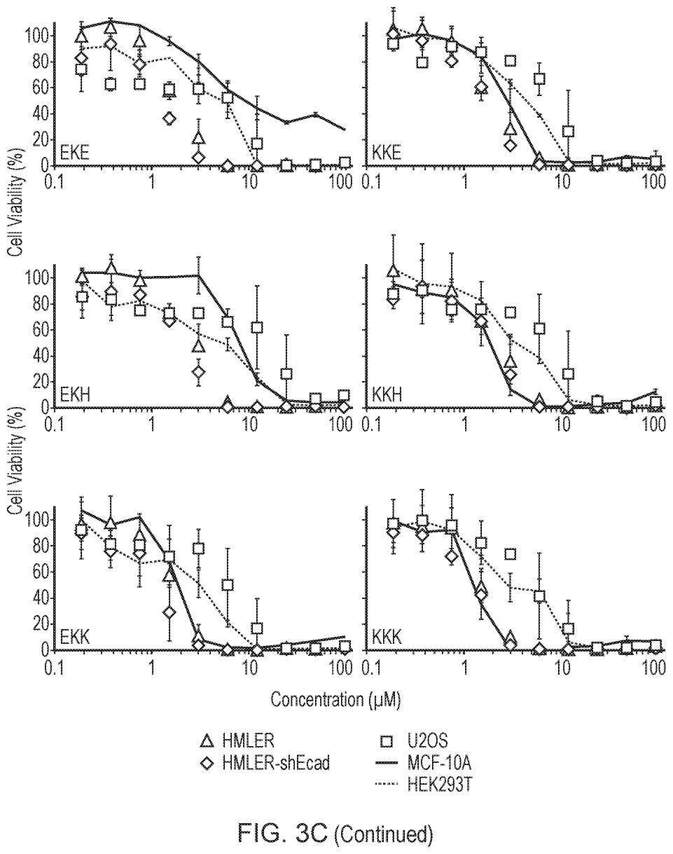

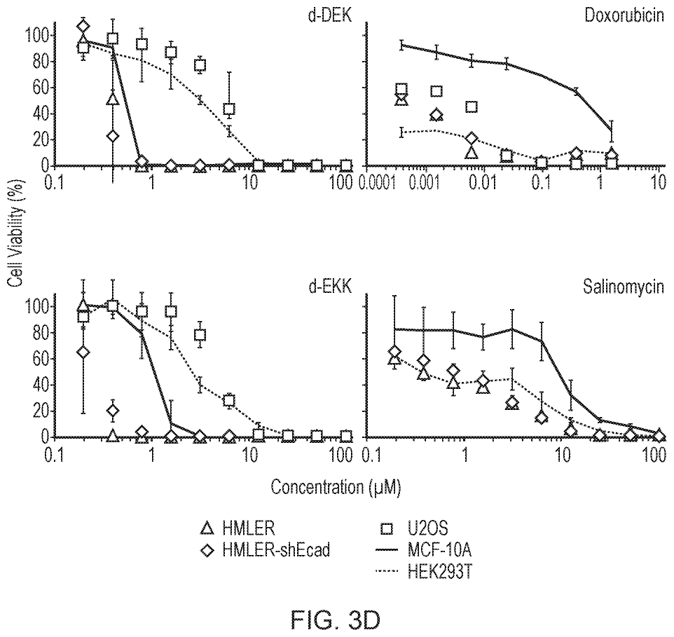

FIG. 3 shows the in vitro cytotoxic dose response of two clinically used anticancer drugs doxorubicin and salinomycin, in comparison to two selected D-form anticancer peptides (D-form DEK, and D-form EEK), and 36 leucine-rich anticancer peptides against different human cell lines, e.g. HMLER (triangles), HMLER-shEcad (diamonds), MCF-10A (solid lines), U2OS (squares), and HEK293T (dotted lines).

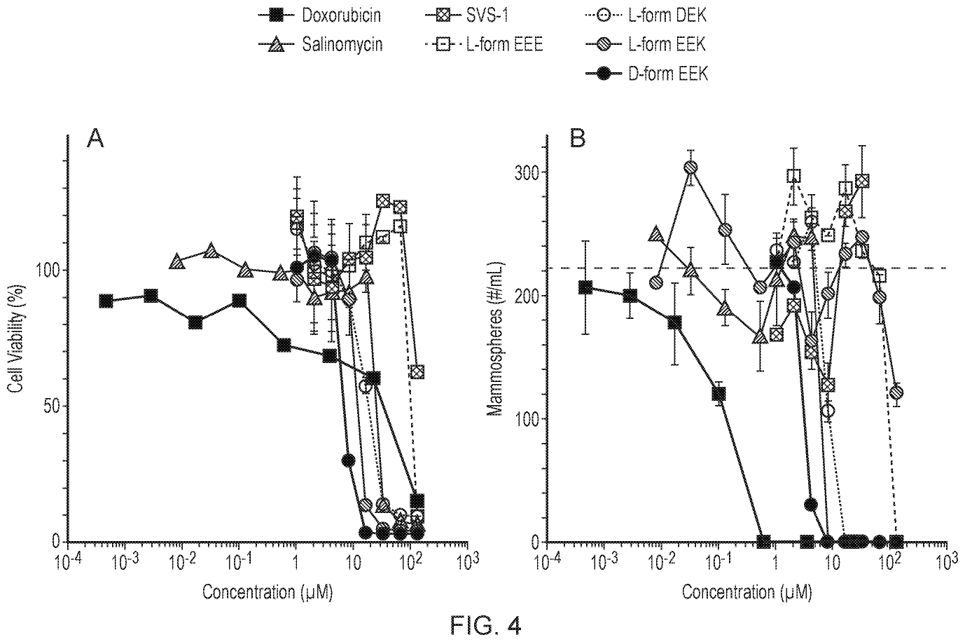

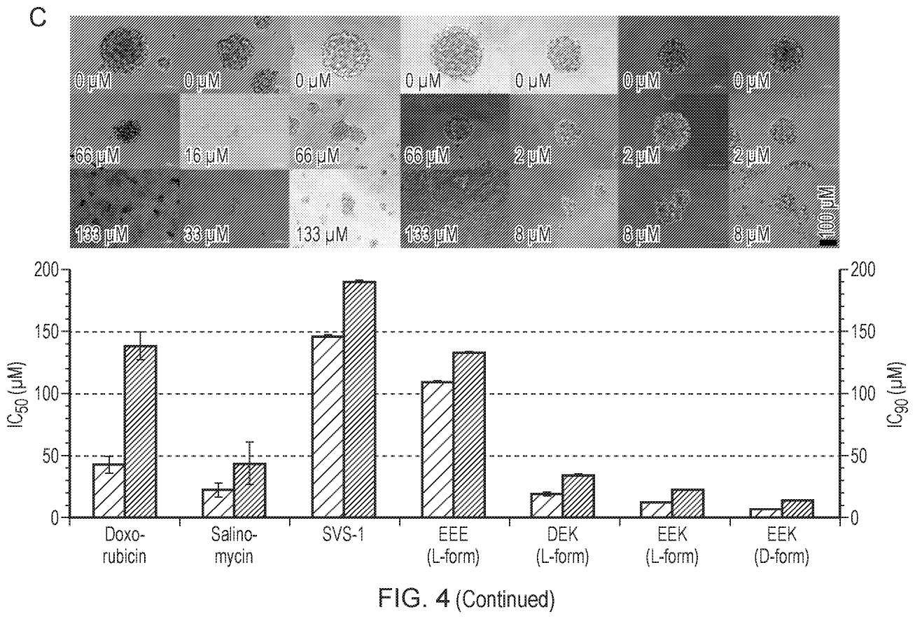

FIG. 4 shows the tumoursphere (HMLER-shEcad cells) in vitro cytotoxicity and dose response of doxorubicin (filled squares), salinomycin (filled triangles) and the leucine-rich-based anticancer peptides L-form EEE (squares), L-form DEK (circles), L-form EEK (grey circles) and D-form EEK (black circles). A) Cell viability is measured to quantify the potency of the anticancer drugs against tumour cell (HMLER-shEcad) mammospheres. B) Mammosphere population after treatment with the selected anticancer compounds. The dashed line presents the expected negative control without any treatment. C) The measured IC₅₀ (grey bar) and IC₉₀ (black bar) of each anticancer drug and optical microscope images of the mammospheres at specific concentration. The scale bar is 100 μm.

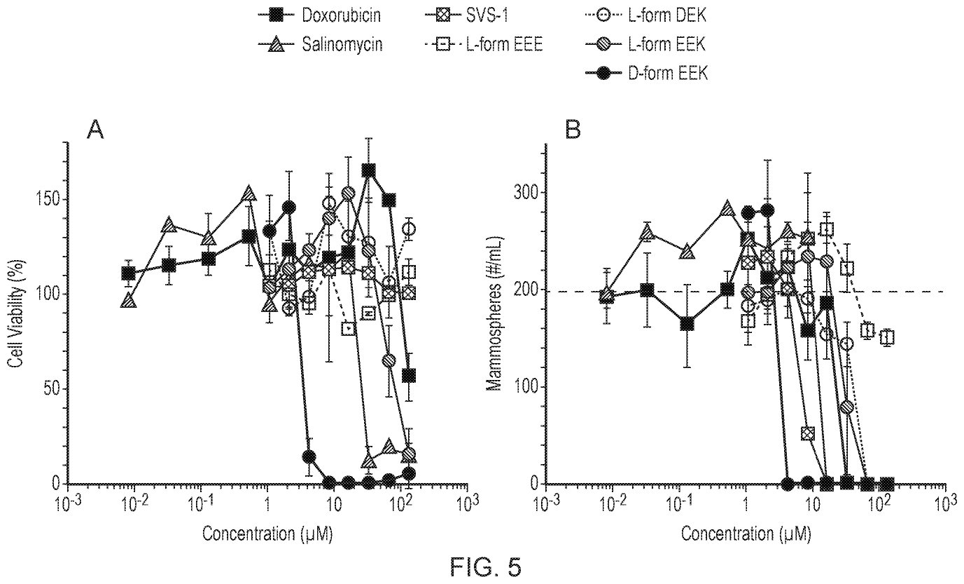

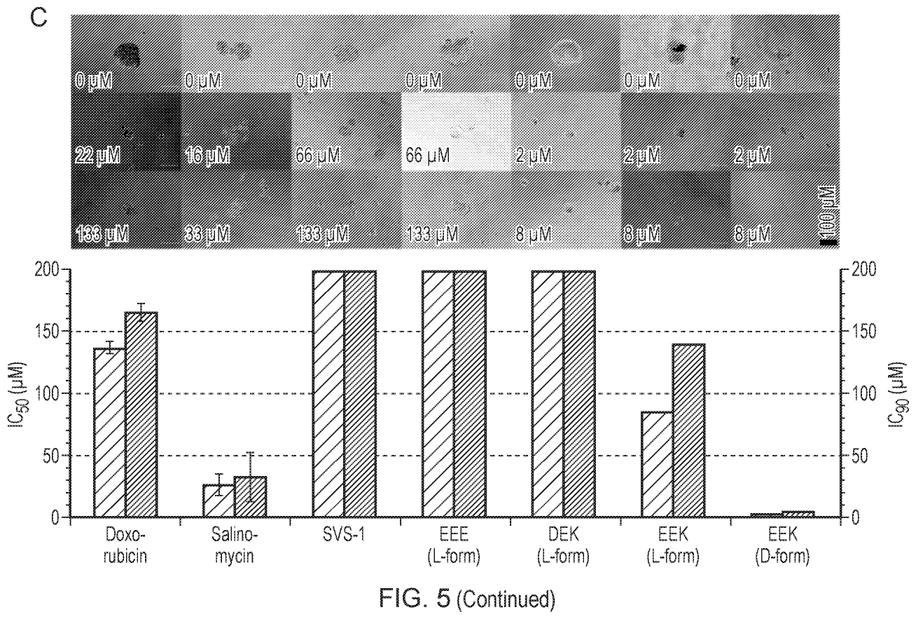

FIG. 5 shows the mammosphere (MCA-10A cells) in vitro cytotoxicity and dose response of doxorubicin (filled squares), salinomycin (filled triangles) and the leucine-rich-based anticancer peptides L-form EEE (squares), L-form DEK (circles), L-form EEK (grey circles) and D-form EEK (black circles). Cell viability is measured to quantify the potency of the anticancer drugs against healthy human breast endothelial cell (MCA-10A) mammospheres. B) Mammosphere population after treatment with the selected anticancer compounds. The dashed line presents the negative control without any treatment. C) The measured IC₅₀ (solid bar) and IC₉₀ (bar) of each anticancer drug and optical microscope images of the mammospheres at specific concentration. The scale bar is 100 μm.

FIG. 6 shows the in vitro cytotoxicity and dose response of doxorubicin, salinomycin, L-form EEK, and D-form EEK against different human cell lines: HMLER (circles), HMLER-shEcad (grey filled circles), U2OS (squares), MCF-10A (black filled circles), and HEK293T (triangles). The shaded regions indicate the ideal compound concentrations that have cell-selectivity towards cancer cell lines with less effect on normal cell lines (MCF-10A and HEK293T).

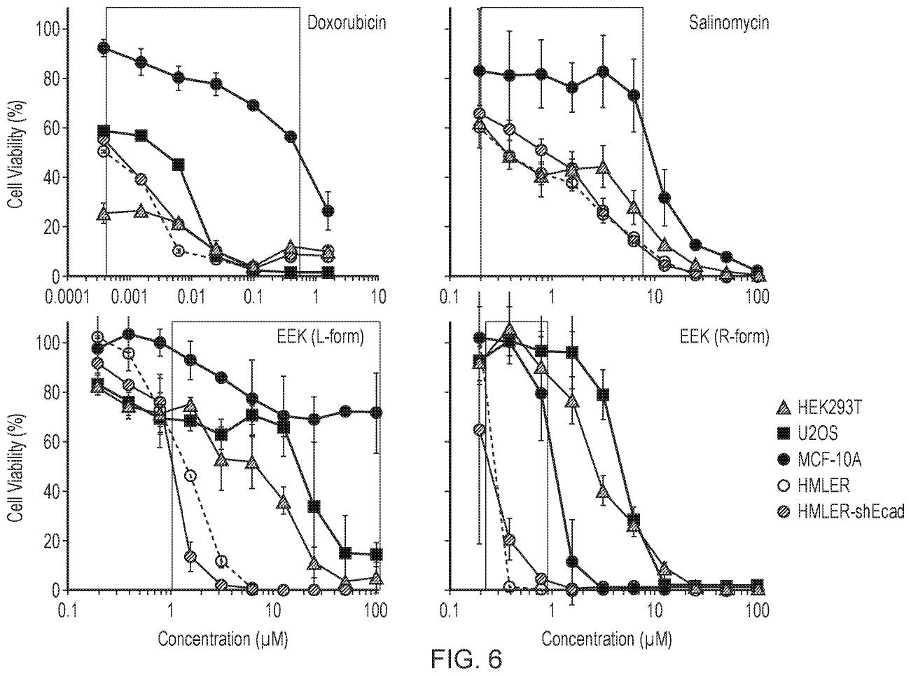

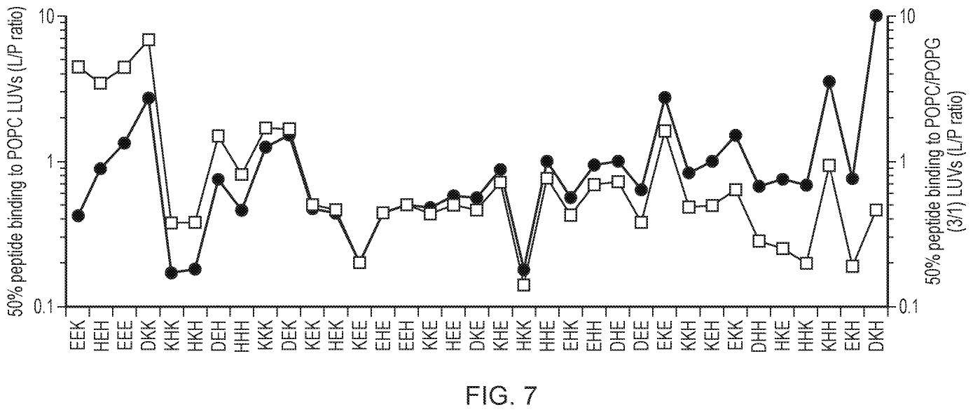

FIG. 7 shows the results of the tryptophan fluorescence binding assay. It shows the lipid concentration at which 50% of the peptide binds to either a single lipid species POPC liposome (circles), or mixed lipid species POPC:POPG (ratio 3:1, squares) liposomes. In brief, 50 μM peptides were fixed and incubated with titrated POPC vesicles (black) or 3POPC/1POPG vesicles (grey) at concentrations of 0, 12.5, 25, 50, 100, 250, 500, 1000, 2500, and 5000 μM in phosphate buffered saline (1×, pH 7.4). The lipid concentration that causes 50% peptide binding was determined using a tryptophan fluorescent binding assay and the values are shown as lipid per peptide. This data demonstrates that the peptides of the invention can distinguish between a neutral vesicle (POPC) and a charged one (POPC/POPG), the latter acting as a model for a cancer cell (Warburg effect).

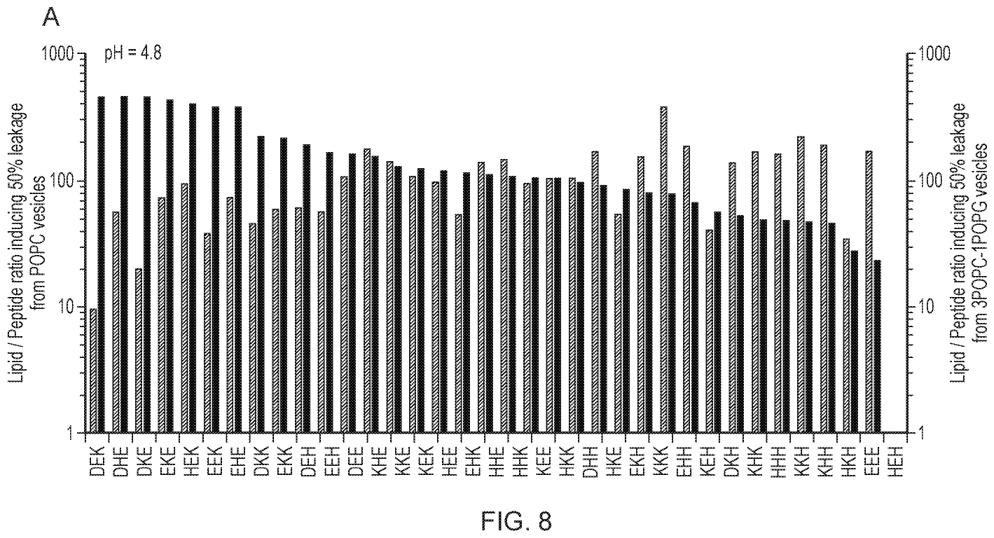

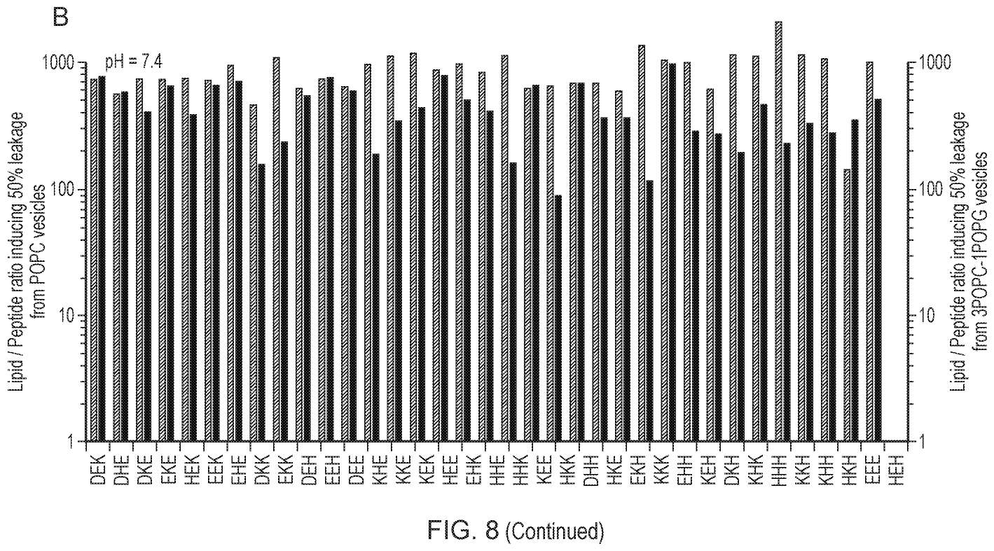

FIG. 8 shows the peptide concentration that causes 50% leakage of ANTS/DPX dyes from liposomes. In brief, 0.5 mM POPC vesicles (grey) or POPC:POPG vesicles (ratio 3:1, black) were incubated with peptide concentrations of 0, 0.02, 0.04, 0.08, 0.16, 0.32, 0.64, 1.25, 2.5, 5, 10, and 20 μM in each A) hydrochloric acid-adjusted phosphate buffered saline (1×, pH 4.8) and B) phosphate buffered saline (1×, pH 7.4). The strength of peptide-induced dye leakage is reported as the number of lipids per peptide (a high number signifies a peptide that is more potent at disrupting the lipid membrane).

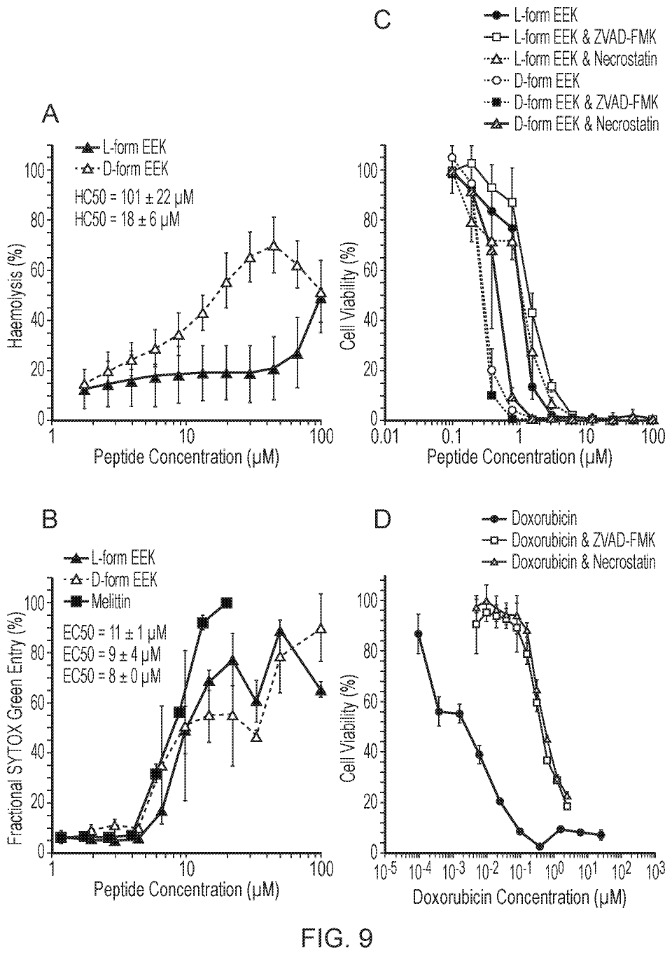

FIG. 9 shows the mechanism of action of the leucine-rich ACPs. A) Hemolytic activity of L-form EEK (black triangles) and D-form EEK (grey triangles) against human red blood cells. B) Peptide-induced high-affinity nucleic acid stain (SYTOX green) entry into HeLa cell line with titrated peptide concentrations: L-form EEK (black triangles), D-form EEK (grey triangles), and melittin (squares) as a positive control. C) HMLER-shEcad (human mammary endothelial cancer stem cells) cell viability in the presence of L-form EEK (black circles) and D-form EEK (grey circles) and co-incubated together with necrostatin (inhibitor of necroptosis) and ZVAD-FMK (inhibitor of apoptosis). D) Viability of HMLER-shEcad cells treated with doxorubicin (cirles), and doxorubicin in combination with 5 μM capase inhibitor z-VAD-FMK (square), and doxorubicin with 20 μM necrostatin-1 (triangles)

FIG. 10 shows the synthesis strategy for conjugation of the present ACPs with copper-based small molecule anticancer drugs.

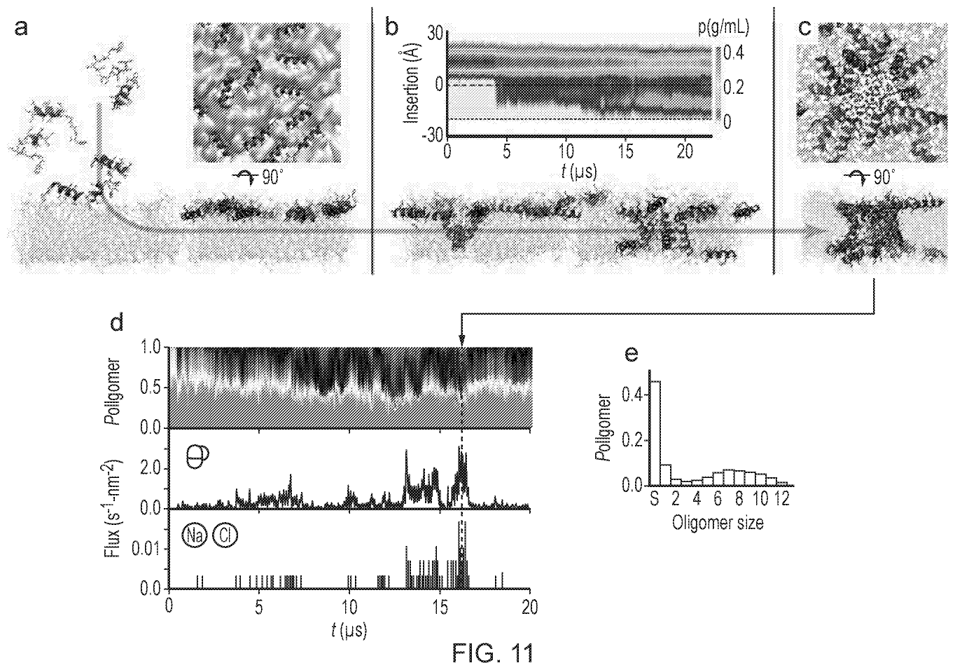

FIG. 11 shows that atomic detail ACP membrane pore structures and membrane perforation mechanism. Molecular dynamics simulations reveal the full atomic details of a, spontaneous ACP membrane adsorption. b, insertion and c, pore formation (shown is a large, heterogeneous, fully water-filled EEK pore). d,e Bound peptides form an ensemble of transient pores of 2-16 peptides (top) that conduct both water (middle) and ions (bottom) across the membrane.

### EXAMPLE 1

### Materials and Methods

### Peptide Synthesis and Purification

Peptides were solid-phase synthesized and purified to 98% purity. Peptide purity and identity were confirmed by HPLC and ESI mass spectrometry. The N-terminus was a free amine group and the C-terminus was either a free carboxyl group or amidated.

### Liposome Production

The lipids 1-palmitoyl-2-oleoyl-sn-glycero-3-phosphocholine (POPC), 1-palmitoyl-2-oleoyl-snglycero-3-phospho-(1′-rac-glycerol) (POPG) were purchased from Avanti Polar Lipids and dissolved in chloroform. Large unilamellar vesicles (LUVs) were produced by extrusion through 100 nm pore filter using an extruder and filters purchased from Avanti Polar Lipids.

### Cell Lines and Cell Culture Conditions

HMLER (human mammary endothelial cancer cells), HMLER-shEcad (human mammary endothelial cancer stem cells), and MCF-10A (healthy human mammary endothelial) cells were maintained in Mammary Epithelial Cell Growth Medium (MEGM) with supplements and growth factors: bovine pituitary extract (BPE), hydrocortisone, human epidermal growth factor (hEGF), insulin, and gentamicin/amphotericin-B. HEK293T (human embryonic kidney cell), and U2OS (Homo sapiens bone osteosarcoma) cells were maintained in Dulbecco's Modified Eagle's Medium (DMEM) with a final concentration of 10% fetal bovine serum. The cells were grown in T75 flask at 310 K in a humidified atmosphere containing 5% CO₂.

### Cytotoxicity Assay

The colourimetric MTT (3-(4,5-dimethylthiazol-2-yl)-2,5-diphenyltetrazolium bromide) assay was used to determine the toxicity of the anticancer peptides and conventional anticancer drugs. 5×10³ cells were seeded in each well of a 96-well microplate. The cells were incubated overnight. Elevated concentrations of the compounds (0, 0.1, 0.2, 0.4, 0.8, 1.6, 3.1, 6.3, 12.5, 25, 50 and 100 μM) were added and incubated for 72 hr with a total volume 200 μL. The stock solutions of the compounds were prepared as 5 mM solutions in DMSO and diluted using media or in pure water. The final concentration of DMSO in each well was either 0.5% or 0% and this amount was present in the untreated control. After 72 hr, 20 μL of a 4 mg/mL solution of MTT in PBS was added to each well, and the plate was incubated for an additional 4 hr. The MEGM/MTT mixture was aspirated and 100 μL of DMSO was added to dissolve the resulting purple formazan crystals. The absorbance of the solutions in each well was read at 550 nm wavelength. Absorbance values were normalized to either DMSO-containing or non DMSO-containing control wells and plotted as concentration of test compound versus % cell viability. IC₅₀ values were interpolated from the resulting dose dependent curves. The reported IC₅₀ values are the average of two independent experiments, each consisting of six replicates per concentration level (overall n=12). The IC₅₀ values for 36 leucine-rich-based peptides were average of two independent experiments (overall n=2).

### Tumoursphere Formation and Viability Assay

HMLER-shEcad cells (5×10³) were plated in ultralow-attachment 96-well plates (Corning) and incubated in MEGM supplemented with B27 (Invitrogen), 20 ng/mL EGF, and 4 μg/mL heparin (Sigma) for 5 days. Studies were conducted in the absence and presence of anticancer peptides, doxorubicin, and salinomycin. Mammospheres treated with anticancer peptides, doxorubicin, and salinomycin were counted and imaged using an inverted based reagent, TOX8 (Sigma). After incubation for 16 hr, the fluorescence of the solutions was read at 590 nm (λₑₓ=560 nm). Viable mammospheres reduce the amount of the oxidized TOX8 and concurrently increases the amount of the fluorescent TOX8 intermediate, indicating the degree of mammosphere cytotoxicity caused by the test compound. Fluorescence values were normalized to DMSO-containing or non DMSO-containing controls and plotted as concentration of test compound versus % mammosphere viability. IC₅₀ values were interpolated from the resulting dose dependent curves. The reported IC₅₀ values are the average of two independent experiments, each consisting of two replicates per concentration level (overall n=4).

### Tryptophan Fluorescent Binding Assay

Peptides (50 μM) and POPC/POPG LUVs (600 μM) were prepared in 10 mM phosphate buffer (pH 7.0). The solutions were incubated and measured after 60 minutes. Excitation was fixed at 280 nm (slit 9 nm) and emission was collected from 300 to 450 nm (slit 9 nm). The spectra were recorded using a Synergy H1 Hybrid Multi-Mode Reader (FIG. 3A) and Cytation™ 5 Cell Imaging Multi-Mode Reader (FIG. 2) from BioTek and were averaged over 3 scans.

### Liposome Leakage Assay

5 mM ANTS (8-aminonaphthalene-1,3,6-trisulfonic acid, disodium salt) and 12.5 mM DPX (p-xylene-bis-pyridinium bromide) were entrapped in 0.1 μm diameter extruded vesicles with lipids. Gel filtration chromatography using a Sephadex G-100 (GE Healthcare Life Sciences Inc) was used to remove external free ANTS/DPX from LUVs with entrapped contents. LUVs were diluted to 0.5 mM and used to measure the leakage activity by addition of aliquots of peptides. Leakage was measured after 3 h incubation. 10% Triton was used as the positive control to measure the maximum leakage of the vesicle. Fluorescence emission spectra were recorded using excitation and emission wavelength of 350 nm and 510 nm for ANTS/DPX using a BioTek Synergy H1 Hybrid Multi-Mode Reader.

### Hemolysis Assay

Peptides were serially diluted in PBS starting at a concentration of 100 PM. The final volume of peptide in each well was 50 μL. To each well, 50 μL of RBCs in PBS at 2×10⁸ cells/mL was added. As a positive lysis control, 1% triton was used. The mixtures were incubated at 37° C. for 1 hour, after which they were centrifuged at 1000×g for 5 minutes. After centrifugation, 10 μL of supernatant was transferred to 90 μL of DI H₂O in a fresh 96-well plate. The absorbance of released hemoglobin at 410 nm was recorded and the fractional hemolysis was calculated based on the 100% and 0% lysis controls.

### Sytox Green Assay to Measure Cytotoxicity Against Hela Cells

Hela cells were grown to confluency in T-75 flasks in complete DMEM (10% FBS). The day prior to cytotoxicity experiments, cells were trypsinized, removed from the flask, and pelleted at 1300 rpm. The trypsin and spent media were discarded and the cells were resuspended in complete DMEM. The cell count was obtained using a cell counter. The cells were then seeded at a density of 10,000 cells/well in a 96-well tissue-culture plate. Next day, in a separate 96-well plate, peptide was serially diluted in complete DMEM (10% with FBS) and 0.1% sytox green starting at a concentration of 100 M (1st), 67 μM (2nd) which was followed by 2:3 serial dilutions. The final volume of peptide in each well was 100 μL. To perform the cytotoxicity assay, media was removed from the wells and replaced with the peptide/DMEM/sytox green solutions. No peptide and 20 μM MelP5 were used as negative and positive controls, respectively. The plate was read for fluorescence every 5 minutes for an hour with an excitation wavelength of 504 nm and emission wavelength 523 nm. Cytotoxicity was calculated based on the 100% and 0% lysis controls based on the sytox green entered in to the cells due to cell wall destabilization.

### Molecular Dynamics Simulations and Analysis

Unbiased all-atom MD simulations were performed and analyzed using GROMACS 2018.3 (www.gromacs.org), Hippo BETA (http://www.biowerkzeug.org), and VMD (http://www.ks.uiuc.edu/Research/vmd/).

Extended peptide structures were generated using Hippo BETA. These initial structures were relaxed via 200 Monte Carlo steps, with water treated implicitly using a Generalized Born solvent. After relaxation, the peptides were placed in atomic detail peptide/lipid/water systems containing model membranes with 100 mM K and Cl ions using CHARMM-GUI (http://www.charmm-qui.org/). Protein folding simulations were equilibrated for 10 ns with applying position restraints to the peptide. For pore-forming simulations single peptides were allowed to fold onto the bilayer for ˜600 ns. Once a stable surface state had been obtained, subsequently the systems were multiplied 4×4 in the x and y (but not z) directions, resulting in a system with 16 peptides. When starting with peptides from both sides of the membrane, the initial structure had one peptide in the upper and one in the lower leaflet. The large system was then constructed by multiplexing 3×3 to obtain an 18-peptide simulation box. MD simulations were performed with GROMACS 2018.3 using the CHARMM36 force field, in conjunction with the TIP3P water model. Electrostatic interactions were computed using PME, and a cut-off of 10 Å was used for van der Waals interactions. The integration time-step was 2 fs and neighbour lists were updated every 5 steps. All simulations were performed in the NPT ensemble, without any restraints or biasing potentials. Water and the protein were each coupled separately to a heat bath with a time constant τ_{T=}0.5 ps using velocity rescale temperature coupling. The atmospheric pressure of 1 bar was maintained using weak semi-isotropic pressure coupling with compressibility κ_{z}=κ_{xy}=4.6·10⁻⁵ bar⁻¹ and time constant τ_{P}=1 ps.

### Oligomer Population Analysis

In order to reveal the most populated pore assemblies during the simulations, a complete list of all oligomers was constructed for each trajectory frame. An oligomer of order n was considered any set of n peptides that are in mutual contact, defined as a heavy-atom (N, C, O) minimum distance of <3.5 Å. Frequently, this definition overcounts the oligomeric state due to numerous transient surface bound (S-state) peptides that are only loosely attached to the transmembrane inserted peptides that make up the core of the oligomer. These S-state peptides frequently change position or drift on and off the stable part of the pore. To focus the analysis on true longer-lived TM pores, a cut-off criterion of 75° was introduced for the tilt angle τ of the peptides. Any peptide with τ≥75° was considered in the S-state and removed from the oligomeric analysis. This strategy greatly reduced the noise in the oligomeric clustering algorithm by focusing on the true longer-lived pore structures. Population plots of the occupation percentage of oligomer n multiplied by its number of peptides n, were then constructed. These reveal how much peptide mass was concentrated in which oligomeric state during the simulation time.

### Permutational Cluster Analysis

All oligomers of the same order n were conformationally clustered using a clustering algorithm with a backbone RMSD similarity cutoff criterion of 4 Å. Since each oligomer could be made up of different peptides—or of the same peptides, but in a different order—the clustering compares one oligomer with all n! permutations of peptide arrangements of another oligomer. Permutations were generated using Heap's algorithm. The final RMSD value of the conformational similarity was considered the lowest RMSD value as obtained from the n! permutational comparisons. Clustering results were generally flat, indicating that structures are highly fleeting and dynamical.

### Transmembrane Flux

Water and ion flux through membrane pores was calculated by determining the total instantaneous flux through the whole bilayer patch. Two planes orthogonal to the membrane normal were considered at z=−7 Å and z=+7 Å, with all transition events that cross thoe planes counted. The flux was then obtained by dividing the transition counts by the area of the membrane patch and the elapsed time for each trajectory frame. Curves were subsequently smoothed by averaging over 1000 frames.

### EXAMPLE 2

### Peptide Rationale

Table 1 below comprises 36 peptides which fall within the scope of the present disclosure.

TABLE 1

| | | | | Iso- | | |
|---|---|---|---|---|---|---|
| | | MW | Net | electric | ΔG_{interfacial} | Hydrophobic |
| Name | Sequence† | (g/mol) | Charge | Point | (kcal/mol) | Moment |
| DEE | GLLDLLELLL | 1625 | −2 | 3.69 | −2.31 | 4.94 |
| | EAAG | | | | | |
| EEE | GLLELLELLLE | 1639 | −2 | 3.85 | −1.52 | 5.56 |
| | AAG | | | | | |
| HEE | GLLHLLELLL | 1647 | −1 | 5.26 | −2.58 | 4.72 |
| | EAAG | | | | | |
| KEE | GLLKLLELLL | 1638 | 0 | 7 | −2.55 | 4.74 |
| | EAAG | | | | | |
| DHE | GLLDLLHLLL | 1633 | −1 | 5.17 | −3.37 | 3.95 |
| | EAAG | | | | | |
| EHE | GLLELLHLLL | 1647 | −1 | 5.26 | −2.58 | 4.62 |
| | EAAG | | | | | |
| HHE | GLLHLLHLLL | 1655 | 0 | 7.96 | −3.64 | 3.73 |
| | EAAG | | | | | |
| KHE | GLLKLLHLLL | 1646 | 1 | 10.12 | −3.61 | 3.75 |
| | EAAG | | | | | |
| DKE | GLLDLLKLLL | 1624 | 0 | 6.92 | −3.34 | 3.98 |
| | EAAG | | | | | |
| EKE | GLLELLKLLL | 1638 | 0 | 7 | −2.55 | 4.65 |
| | EAAG | | | | | |
| HKE | GLLHLLKLLL | 1646 | 1 | 10.12 | −3.61 | 3.76 |
| | EAAG | | | | | |
| KKE | GLLKLLKLLL | 1637 | 2 | 10.73 | −3.58 | 3.78 |
| | EAAG | | | | | |
| DEH | GLLDLLELLL | 1633 | −1 | 5.17 | −3.37 | 3.94 |
| | HAAG | | | | | |
| EEH | GLLELLELLL | 1647 | −1 | 5.26 | −2.58 | 4.54 |
| | HAAG | | | | | |
| HEH | GLLHLLELLL | 1655 | 0 | 7.96 | −3.64 | 3.76 |
| | HAAG | | | | | |
| KEH | GLLKLLELLL | 1646 | 1 | 10.12 | −3.61 | 3.78 |
| | HAAG | | | | | |
| DHH | GLLDLLHLLL | 1641 | 0 | 7.96 | −4.43 | 2.93 |
| | HAAG | | | | | |
| EHH | GLLELLHLLL | 1655 | 0 | 7.96 | −3.64 | 3.58 |
| | HAAG | | | | | |
| HHH | GLLHLLHLLL | 1663 | 1 | 14 | −4.7 | 2.73 |
| | HAAG | | | | | |
| KHH | GLLKLLHLLL | 1654 | 2 | 14 | −4.67 | 2.75 |
| | HAAG | | | | | |
| DKH | GLLDLLKLLL | 1632 | 1 | 10.12 | −4.4 | 2.96 |
| | HAAG | | | | | |
| EKH | GLLELLKLLL | 1646 | 1 | 10.12 | −3.61 | 3.61 |
| | HAAG | | | | | |
| HKH | GLLHLLKLLL | 1654 | 2 | 14 | −4.67 | 2.76 |
| | HAAG | | | | | |
| KKH | GLLKLLKLLL | 1645 | 3 | 14 | −4.64 | 2.78 |
| | HAAG | | | | | |
| DEK | GLLDLLELLL | 1624 | 0 | 6.92 | −3.34 | 3.97 |
| | KAAG | | | | | |
| EEK | GLLELLELLL | 1638 | 0 | 7 | −2.55 | 4.57 |
| | KAAG | | | | | |
| HEK | GLLHLLELLL | 1646 | 1 | 10.12 | −3.61 | 3.78 |
| | KAAG | | | | | |
| KEK | GLLKLLELLL | 1637 | 2 | 10.73 | −3.58 | 3.8 |
| | KAAG | | | | | |
| DHK | GLLDLLHLLL | 1632 | 1 | 10.12 | −4.4 | 2.96 |
| | KAAG | | | | | |
| EHK | GLLELLHLLL | 1646 | 1 | 10.12 | −3.61 | 3.61 |
| | KAAG | | | | | |
| HHK | GLLHLLHLLL | 1654 | 2 | 14 | −4.67 | 2.76 |
| | KAAG | | | | | |
| KHK | GLLKLLHLLL | 1645 | 3 | 14 | −4.64 | 2.78 |
| | KAAG | | | | | |
| DKK | GLLDLLKLLL | 1623 | 2 | 10.73 | −4.37 | 2.99 |
| | KAAG | | | | | |
| EKK | GLLELLKLLL | 1637 | 2 | 10.73 | −3.58 | 3.63 |
| | KAAG | | | | | |
| HKK | GLLHLLKLLL | 1645 | 3 | 14 | −4.64 | 2.79 |
| | KAAG | | | | | |
| KKK | GLLKLLKLLL | 1636 | 4 | 14 | −4.61 | 2.81 |
| | KAAG | | | | | |

†N-terminus is free, C-terminus: W-NH2. Shown are computational predictions of the isoelectric point, the estimated interfacial binding free energy and the hydrophobic moment.

The interfacial binding free energy is a measure of how likely the peptide is to bind to a membrane and the hydrophobic moment is a measure of how evenly the hydrophobic residues are distributed around the surface of the peptide in its helical, membrane inserted, conformation.

An additional tryptophan was introduced at the C-terminus in order to quantify the peptide concentration. The charged carboxylic C-terminus (—CO₂⁻) was also modified to a neutral amide group (—NH₂) to further promote membrane penetration. The peptides are designed such that the charged residues are located on the same polar face of the helical structure. Therefore, the charge distribution may affect the peptides' hydrophobic moment, pKa, binding strength onto the cancer cell membrane, and ultimately the structure of the peptide assembly within the cancer cell membrane (FIG. 1A). Many pH-dependent peptides with biomedical applications targeting cancer have a pKa ˜4.0. This may stem from the slightly more acidic microenvironment of cancer cells, which is due to the Warburg effect. It is therefore believed that the cancer cell membrane can protonate negative amino acids of the present invention, and result in pH-triggered membrane activity (FIG. 1B and Table 1).¹⁶⁻¹⁹

All 36 leucine-rich peptide sequences were synthesised as the L-form. ΔG_{interfacial} represents the binding free energy of peptide partition between water and the membrane interface. ΔG_{interfacial} and hydrophobic moment were estimated using the Wimley-White hydrophobicity scale using the MPEx software. The binding free energy is the energy released upon binding of a peptide to a membrane. At 0 the peptide is 50% in water 50% on the membrane, negative it preferentially inserts, positive it prefers the aqueous phase. The hydrophobic moment is a measure of how the hydrophobic residues are spaced around the helical wheel; a large moment they're all on one side, a low moment they're evenly spaced around. Large moments are better for surface binding (i.e. the hydrophobic face dips into the bilayer and the hydrophilic face points to the water).

### EXAMPLE 3

### Cytotoxicity and Efficacy

The peptides were screened against several different human cell lines and their cytotoxicity were determined. Cell lines utilised include MCF-10A (human breast epithelial cell), HMLER (human breast cancer bulk cell), HMLER-shEcad (human breast cancer stem cell), HEK293T (human embryonic kidney cell), and U2OS (human bone osteosarcoma). It emerged that the peptides are as potent as conventional cancer drugs that can eliminate the cancer cells with low micromolar concentration, and many have high selectivity toward cancer cell lines (FIG. 2 and Table 1). Although both doxorubicin and salinomycin also have selectivity for cancerous HMLER over healthy MCF-10A cells, they are both significantly more toxic to HEK293T cells. In addition, both drugs are much less efficient at clearing cancer cells grown as three-dimensional mammospheres, which is considered a far more accurate in vitro model for solid tumours at present. The half maximal inhibitory concentrations (IC₅₀) of doxorubicin and salinomycin against two-dimensional HMLER-shEcad are 2.5±0.3 nM and 370±0.5 nM, respectively, however in mammospheres, a more realistic three-dimensional cell culture model that is much more relevant to the in vivo condition, these values drop to 43±6 μM and 22±5 μM respectively, a 1,700-fold decrease in activity for doxorubicin and 63 times for salinomycin. See Table 2 below and FIG. 3. In comparison, the selected sequence EEK (GLLELLELLLKAAGW), and its D-form peptide are effective against both two-dimensional as well as three-dimensional mammosphere tumor models, with nano- to low micro-molar activity against two-dimensional cultures of HMLER, HMLER-shEcad, and U2OS cell and 7-13 μM activity against mammosphere. See FIGS. 4 to 6.

All data points were performed in duplicate. The selected D-form peptides, conventional anticancer drugs, EEK peptide and 25B2 peptide were repeated six times. The †N-terminus is free, C-terminus: —WNH2.

TABLE 2

| | | | HMLER- | IC₅₀ (μM) | | |
|---|---|---|---|---|---|---|
| Name | Sequence† | HMLER | shEcad | MCF-10A | U20S | HEK293T |
| DEE | GLLDLLELLL | 5.55 ± 0.35 | 6.05 ± 2.76 | 8.40 ± 1.56 | 61.00 ± 1.41 | 9.91 ± 0.44 |
| | EAAG | | | | | |
| EEE | GLLELLELLL | 6.25 ± 1.77 | 10.55 ± 3.46 | 200 ± 0 | 78.75 ± 5.30 | 50.00 ± 0 |
| | EAAG | | | | | |
| HEE | GLLHLLELLL | 6.50 ± 0.71 | 4.95 ± 0.49 | 58 ± 41 | 47.50 ± 11 | 10.25 ± 0.2 |
| | EAAG | | | | | |
| KEE | GLLKLLELLL | 3.75 ± 0.78 | 2.05 ± 0.21 | 200 ± 0 | 49.38 ± 13.26 | 11.10 ± 1.84 |
| | EAAG | | | | | |
| DHE | GLLDLLHLLL | 3.75 ± 0.92 | 2.80 ± 0.14 | 22.65 ± 4.31 | 17.25 ± 1.06 | 11.05 ± 1.91 |
| | EAAG | | | | | |
| EHE | GLLELLHLLL | 3.90 ± 0.28 | 2.55 ± 0.07 | 107 ± 37 | 18.25 ± 1.06 | 13.00 ± 0.00 |
| | EAAG | | | | | |
| HHE | GLLHLLHLLL | 16.70 ± 2.26 | 10.75 ± 0.78 | 200 ± 0 | 53.25 ± 5.30 | 18.35 ± 3.75 |
| | EAAG | | | | | |
| KHE | GLLKLLHLLL | 3.70 ± 0.14 | 2.92 ± 0.17 | 20.85 ± 5.87 | 53 ± 2.83 | 26.00 ± 2.83 |
| | EAAG | | | | | |
| DKE | GLLDLLKLLL | 2.10 ± 0 | 1.57 ± 0.33 | 4.75 ± 0.49 | 9.93 ± 0.25 | 7.80 ± 1.41 |
| | EAAG | | | | | |
| EKE | GLLELLKLLL | 1.80 ± 0.28 | 1.30 ± 0.14 | 8.80 ± 0 | 8.00 ± 0.71 | 5.15 ± 0.21 |
| | EAAG | | | | | |
| HKE | GLLHLLKLLL | 2.70 ± 0.28 | 1.90 ± 0.42 | 7.60 ± 0.42 | 15.95 ± 0.78 | 5.15 ± 0.64 |
| | EAAG | | | | | |
| KKE | GLLKLLKLLL | 2.05 ± 0.64 | 1.70 ± 0 | 2.90 ± 0.57 | 12.75 ± 1.20 | 4.40 ± 0.14 |
| | EAAG | | | | | |
| DEH | GLLDLLELLL | 5.35 ± 1.91 | 3.60 ± 0.42 | 20.25 ± 6.72 | 36.48 ± 3.92 | 10.50 ± 4.67 |
| | HAAG | | | | | |
| EEH | GLLELLELLL | 3.30 ± 0.14 | 3.60 ± 0.85 | 167 ± 47 | 19.60 ± 3.96 | 12.90 ± 0.14 |
| | HAAG | | | | | |
| HEH | GLLHLLELLL | 28.60 ± 10.75 | 10.95 ± 1.06 | 200 ± 0 | 106 ± 14 | 8.80 ± 1.70 |
| | HAAG | | | | | |
| KEH | GLLKLLELLL | 3.30 ± 0.42 | 2.35 ± 0.07 | 20.00 ± 0 | 49.25 ± 5.30 | 5.65 ± 0.07 |
| | HAAG | | | | | |
| DHH | GLLDLLHLLL | 21.45 ± 2.47 | 11.90 ± 0.14 | 200 ± 0 | 55.85 ± 18.88 | 6.40 ± 0.42 |
| | HAAG | | | | | |
| EHH | GLLELLHLLL | 25.35 ± 2.76 | 16.05 ± 3.18 | 150 ± 71 | 117 ± 7 | 5.90 ± 0.14 |
| | HAAG | | | | | |
| HHH | GLLHLLHLLL | 21.10 ± 6.93 | 9.50 ± 0.99 | 79.00 ± 11.31 | 185 ± 7 | 30.60 ± 4.81 |
| | HAAG | | | | | |
| KHH | GLLKLLHLLL | 5.20 ± 0.71 | 4.45 ± 0.49 | 10.35 ± 0.49 | 39.75 ± 5.30 | 7.75 ± 0.78 |
| | HAAG | | | | | |
| DKH | GLLDLLKLLL | 3.45 ± 0.07 | 2.80 ± 0.28 | 7.05 ± 0.21 | 21.13 ± 3.01 | 6.15 ± 1.34 |
| | HAAG | | | | | |
| EKH | GLLELLKLLL | 2.75 ± 0.07 | 2.18 ± 0.31 | 7.90 ± 0.42 | 23.83 ± 0.81 | 6.65 ± 1.34 |
| | HAAG | | | | | |
| HKH | GLLHLLKLLL | 3.45 ± 0.21 | 3.10 ± 0.57 | 6.10 ± 0.00 | 18.88 ± 1.24 | 5.70 ± 0.99 |
| | HAAG | | | | | |
| KKH | GLLKLLKLLL | 2.40 ± 0.85 | 2.20 ± 0.42 | 1.75 ± 0.35 | 13.30 ± 0.99 | 4.00 ± 0.28 |
| | HAAG | | | | | |
| DEK | GLLDLLELLL | 1.14 ± 0.52 | 0.70 ± 0.07 | 145 ± 78 | 19.88 ± 2.65 | 6.80 ± 0.85 |
| | KAAG | | | | | |
| EEK | GLLELLELLL | 1.10 ± 0.14 | 1.08 ± 0.18 | 200 ± 0 | 32.88 ± 4.07 | 8.25 ± 0.64 |
| | KAAG | | | | | |
| HEK | GLLHLLELLL | 2.35 ± 1.06 | 3.45 ± 2.19 | 29.80 ± 7.35 | 143.20 ± 80.33 | 6.95 ± 1.63 |
| | KAAG | | | | | |
| KEK | GLLKLLELLL | 1.60 ± 0.14 | 1.35 ± 0.07 | 1.55 ± 0.07 | 8.15 ± 0.49 | 3.20 ± 0.14 |
| | KAAG | | | | | |
| DHK | GLLDLLHLLL | 1.75 ± 0.49 | 1.02 ± 0.12 | 5.05 ± 1.34 | 20.45 ± 2.05 | 7.30 ± 0.42 |
| | KAAG | | | | | |
| EHK | GLLELLHLLL | 1.35 ± 0.35 | 0.71 ± 0.13 | 3.60 ± 0.99 | 25.00 ± 0 | 5.35 ± 0.07 |
| | KAAG | | | | | |
| HHK | GLLHLLHLLL | 3.15 ± 0.07 | 1.75 ± 0.21 | 5.60 ± 0.14 | 12.65 ± 1.91 | 5.25 ± 1.48 |
| | KAAG | | | | | |
| KHK | GLLKLLHLLL | 2.59 ± 0.92 | 1.40 ± 0.14 | 1.81 ± 0.05 | 12.88 ± 1.24 | 3.55 ± 0.78 |
| | KAAG | | | | | |
| DKK | GLLDLLKLLL | 1.95 ± 0.35 | 1.20 ± 0.14 | 2.57 ± 0.52 | 10.40 ± 1.70 | 3.40 ± 0.28 |
| | KAAG | | | | | |
| EKK | GLLELLKLLL | 1.72 ± 0.21 | 1.19 ± 0.40 | 1.70 ± 0.11 | 12.20 ± 1.84 | 3.35 ± 0.49 |
| | KAAG | | | | | |
| HKK | GLLHLLKLLL | 3.40 ± 0.71 | 2.60 ± 0.42 | 2.18 ± 0.39 | 12.23 ± 0.11 | 9.50 ± 0.99 |
| | KAAG | | | | | |
| KKK | GLLKLLKLLL | 1.57 ± 0.24 | 1.35 ± 0.40 | 1.37 ± 0.24 | 9.65 ± 1.20 | 3.70 ± 0.14 |
| | KAAG | | | | | |
| D-form | | 0.32 ± 0.07 | 0.23 ± 0.04 | 0.55 ± 0.06 | 1.24 ± 0.03 | 1.55 ± 0.37 |
| DHK | | | | | | |
| D-form | | 0.44 ± 0.10 | 0.36 ± 0 | 0.57 ± 0.01 | 5.84 ± 0.04 | 3.28 ± 0.21 |
| DEK | | | | | | |
| D-form | | 0.29 ± 0.01 | 0.29 ± 0.01 | 1.07 ± 0.10 | 4.78 ± 0.02 | 2.82 ± 0.07 |
| EEK | | | | | | |
| Doxo- | Doxo- | (2.5 ± 0.3) | (3.0 ± 0.6) | (6.4 ± 0.2) | (1.5 ± 0.8) | (1.1 ± 0.2) |
| rubicin | rubicin | x10⁻³ | x10⁻³ | x10⁻¹ | x10⁻² | x10⁻⁴ |
| Salin- | Salin- | 0.37 ± 0.08 | 0.92 ± 0.28 | 9.76 ± 2.28 | — | 0.41 ± 0.10 |
| omycin | omycin | | | | | |
| 25B2 | GLDDLAKLL | 8.36 ± 0.61 | 11.70 ± 0.49 | 26.74 ± 3.33 | 54.03 ± 8.43 | 25.20 ± 2.19 |
| | LKLAG | | | | | |

### EXAMPLE 4

### Tryptophan Binding Assay and Liposome Leakage Assay

The peptides of the present disclosure are mostly neutral or anionic and do not contain many positive charges in the sequence (Table 1). The present inventors identified six sequences (FIG. 2 and Table 2) that are highly selective to cancer cell lines and have a negligible effect on MCF-10A (IC₅₀≥100 μM) and relatively low cytotoxicity to HEK293T: EEE, KEE, EHE, EEH, DEK, and EEK. Their net charges are between −2 and 0 with a pKa of 3.85-7.96, and their sequences either contain one positive charge (positively charged N-terminus) or two positive charges (one positively charged N-terminus and one lysine at position 4 or 11). Several studies have shown the cancer cell membranes may have a negatively charged membrane surface.^{20,21} Ishikawa et al. found that the breast cancer cell line MCF-7, which is similar to HMLER, contains a low amount of negatively charged sialic acid on the membrane surface.²⁰ This suggests that the anticancer activity and cell selectivity of the present leucine-rich peptides cannot solely be explained by electrostatic interactions but may also involve charge distribution due to the Warburg effect in the microenvironment of cancer cells. To confirm this hypothesis, the present inventors performed tryptophan binding assays (See Table 3 below and FIG. 7) and ANTS/DPX liposome leakage assay (See Table 4 below and FIG. 8) with two different lipid model vesicles (zwitterionic POPC and anionic 3POPC/1POPG mixture) each at pH 7.4 (physiological condition) and pH 4.8 (weak acid).

Table 3 illustrates the lipid concentration-induced 50% peptide binding onto a liposome. 50 μM peptide was fixed and incubated with titrated lipid (POPC vesicles or 3POPC/1POPG vesicles) at concentrations of 0, 12.5, 25, 50, 100, 250, 500, 1000, 2500, and 5000 μM in phosphate buffered saline (1×, pH 7.4). The lipid concentration that causes 50% peptide binding was determined using tryptophan fluorescent binding assay and the values are shown as lipid per peptide. †N-terminus is free, C-terminus: —W—NH₂.

Table 4 illustrates peptide concentration-induced 50% ANTS/DPX liposome leakage. 0.5 mM POPC and 3POPC/1POPG vesicles were fixed and incubated with titrated peptide concentration (0, 0.02, 0.04, 0.08, 0.16, 0.32, 0.64, 1.25, 2.5, 5, 10, and 20 PM) each in phosphate buffered saline (1×, pH 7.4) and hydrochloric acid-adjusted phosphate buffered saline (1×, pH 4.8). The values are shown as lipid per peptide. †N-terminus is free, C-terminus: —W—NH₂.

TABLE 3

| | | Lipid Concentration- | |
|---|---|---|---|
| | | induced | |
| | | 50% Peptide Binding | |
| | | (L/P) | |
| | | POPC | 3POPC/1POPG |
| Name | Sequence† | Vesicle | Vesicle |
| DEE | GLLDLLELLLEAAG | 0.63 | 0.38 |
| EEE | GLLELLELLLEAAG | 1.33 | 4.50 |
| HEE | GLLHLLELLLEAAG | 0.58 | 0.50 |
| KEE | GLLKLLELLLEAAG | 0.20 | 0.20 |
| DHE | GLLDLLHLLLEAAG | 1.00 | 0.72 |
| EHE | GLLELLHLLLEAAG | 0.44 | 0.44 |
| HHE | GLLHLLHLLLEAAG | 1.00 | 0.75 |
| KHE | GLLKLLHLLLEAAG | 0.88 | 0.71 |
| DKE | GLLDLLKLLLEAAG | 0.56 | 0.46 |
| EKE | GLLELLKLLLEAAG | 2.75 | 1.63 |
| HKE | GLLHLLKLLLEAAG | 0.75 | 0.25 |
| KKE | GLLKLLKLLLEAAG | 0.48 | 0.44 |
| DEH | GLLDLLELLLHAAG | 0.75 | 1.50 |
| EEH | GLLELLELLLHAAG | 0.50 | 0.50 |
| HEH | GLLHLLELLLHAAG | 0.88 | 3.50 |
| KEH | GLLKLLELLLHAAG | 1.00 | 0.50 |
| DHH | GLLDLLHLLLHAAG | 0.67 | 0.28 |
| EHH | GLLELLHLLLHAAG | 0.94 | 0.69 |
| HHH | GLLHLLHLLLHAAG | 0.46 | 0.82 |
| KHH | GLLKLLHLLLHAAG | 3.50 | 0.94 |
| DKH | GLLDLLKLLLHAAG | 10.00 | 0.46 |
| EKH | GLLELLKLLLHAAG | 0.75 | 0.19 |
| HKH | GLLHLLKLLLHAAG | 0.18 | 0.38 |
| KKH | GLLKLLKLLLHAAG | 0.83 | 0.48 |
| DEK | GLLDLLELLLKAAG | 1.50 | 1.67 |
| EEK | GLLELLELLLKAAG | 0.48 | 4.50 |
| HEK | GLLHLLELLLKAAG | 0.44 | 0.46 |
| KEK | GLLKLLELLLKAAG | 0.47 | 0.50 |
| DHK | GLLDLLHLLLKAAG | 0.20 | 0.38 |
| EHK | GLLELLHLLLKAAG | 0.56 | 0.42 |
| HHK | GLLHLLHLLLKAAG | 0.68 | 0.20 |
| KHK | GLLKLLHLLLKAAG | 0.17 | 0.38 |
| DKK | GLLDLLKLLLKAAG | 2.75 | 6.88 |
| EKK | GLLELLKLLLKAAG | 1.50 | 0.63 |
| HKK | GLLHLLKLLLKAAG | 0.18 | 0.14 |
| KKK | GLLKLLKLLLKAAG | 1.25 | 1.70 |

TABLE 4

| | | Peptide Concentration- | | Peptide Concentration- | |
|---|---|---|---|---|---|
| | | induced | | induced | |
| | | 50% ANTS/DPX | | 50% ANTS/DPX | |
| | | leakage | | leakage | |
| | | at pH 7.4 (L/P) | | at pH 4.8 (L/P) | |
| | | POPC | 3POPC/1POPG | POPC | 3POPC/1POPG |
| Name | Sequence† | Vesicle | Vesicle | Vesicle | Vesicle |
| DEE | GLLDLLELLLEAAG | 644 | 597 | 110 | 164 |
| EEE | GLLELLELLLEAAG | 143 | 346 | 35 | 28 |
| HEE | GLLHLLELLLEAAG | 876 | 792 | 100 | 122 |
| KEE | GLLKLLELLLEAAG | 626 | 657 | 97 | 108 |
| DHE | GLLDLLHLLLEAAG | 566 | 581 | 58 | 469 |
| EHE | GLLELLHLLLEAAG | 952 | 706 | 75 | 384 |
| HHE | GLLHLLHLLLEAAG | 842 | 411 | 143 | 113 |
| KHE | GLLKLLHLLLEAAG | 981 | 188 | 183 | 156 |
| DKE | GLLDLLKLLLEAAG | 747 | 411 | 21 | 469 |
| EKE | GLLELLKLLLEAAG | 738 | 657 | 75 | 438 |
| HKE | GLLHLLKLLLEAAG | 692 | 365 | 173 | 94 |
| KKE | GLLKLLKLLLEAAG | 1133 | 346 | 143 | 131 |
| DEH | GLLDLLELLLHAAG | 620 | 548 | 63 | 193 |
| EEH | GLLELLELLLHAAG | 738 | 755 | 58 | 168 |
| HEH | GLLHLLELLLHAAG | 995 | 505 | 173 | 24 |
| KEH | GLLKLLELLLHAAG | 995 | 286 | 188 | 68 |
| DHH | GLLDLLHLLLHAAG | 684 | 692 | 106 | 98 |
| EHH | GLLELLHLLLHAAG | 1043 | 981 | 386 | 80 |
| HHH | GLLHLLHLLLHAAG | 1133 | 469 | 173 | 50 |
| KHH | GLLKLLHLLLHAAG | 1153 | 329 | 227 | 48 |
| DKH | GLLDLLKLLLHAAG | 614 | 274 | 41 | 58 |
| EKH | GLLELLKLLLHAAG | 608 | 365 | 55 | 88 |
| HKH | GLLHLLKLLLHAAG | 1077 | 274 | 193 | 47 |
| KKH | GLLKLLKLLLHAAG | 2119 | 227 | 164 | 49 |
| DEK | GLLDLLELLLKAAG | 747 | 773 | 10 | 469 |
| EEK | GLLELLELLLKAAG | 730 | 657 | 39 | 386 |
| HEK | GLLHLLELLLKAAG | 755 | 386 | 97 | 41. |
| KEK | GLLKLLELLLKAAG | 1173 | 438 | 110 | 126 |
| DHK | GLLDLLHLLLKAAG | 747 | 469 | 47 | 183 |
| EHK | GLLELLHLLLKAAG | 981 | 505 | 55 | 117 |
| HHK | GLLHLLHLLLKAAG | 1133 | 160 | 149 | 111 |
| KHK | GLLKLLHLLLKAAG | 1153 | 193 | 140 | 53 |
| DKK | GLLDLLKLLLKAAG | 463 | 156 | 47 | 227 |
| EKK | GLLELLKLLLKAAG | 1095 | 235 | 60 | 219 |
| HKK | GLLHLLKLLLKAAG | 657 | 90 | 106 | 106 |
| KKK | GLLKLLKLLLKAAG | 1369 | 115 | 156 | 81 |

The results show that the cell-selective peptides do not have any significant binding selectivity and peptide-induced liposome leakage between zwitterionic and anionic vesicles at neutral pH, but four (EHE, EEH, DEK, and EEK) out of the six membrane-selective peptides have relatively higher liposome leakage activity from anionic vesicle at pH 4.8. This suggests that these four peptides are environment-triggered membrane-active peptides that depend on both lipid compositions and pH condition; however, the mechanisms of the other two membrane-selective peptides (EEE and KEE) remain unclear.

### EXAMPLE 5

### Mechanism of Action of the Leucine-Rich Peptides

FIG. 9 shows that the L-form of EEK causes minimal lysis below 90 μM concentrations, well below the ˜10 μM therapeutic concentration. D-form EEK is more lytic. Comparison of the concentration-dependent entry of SYTOX green, a high-affinity nucleic acid stain, into HeLa cells shows that L-form and D-form EEK behaves similar to the potent pore-forming peptide melittin. Together these results demonstrate selective pore formation of cancer cell-plasma membranes as the as the mechanism of action.

FIG. 9C shows that cell viability of HMLER-shEcad cells treated with L or D-form EEK cannot be improved by co-incubation with the necroptosis inhibitor necrostatin, nor by co-incubation with the apoptosis inhibitor z-VAD-FMK, suggesting ACPs trigger necrosis due to pore formation in the plasma membrane. In contrast, FIG. 9D shows that the cell viability of HMLER-shEcad cells treated with doxorubicin can be dramatically improved by co-incubation with either z-VAD-FMK or necrostatin.

Together these results suggest selective pore-formation in cancer cell plasma membranes, resulting in necrosis, as the primary mechanisms of ACP anticancer activity.

### EXAMPLE 6

### APC Pore Structures and Function

Membrane-perforating peptides typically form transient pores that elude experimental determination with current technology. To reveal the molecular mechanisms underpinning membrane perforation we studied folding-partitioning and pore assembly of EEK using unbiased long-timescale atomic detail molecular dynamics simulations. ACPs rapidly absorb and fold onto the membrane interface (FIG. 14a). Subsequently, on timescales of tens of μs, APCs cooperatively insert and translocate across the lipid bilayer, populating both membrane interfaces (FIG. 14b), and form an ensemble of pores (FIG. 14d). Structure analysis reveals highly heterogeneous pore architectures, with the majority made up of 6-10 peptides that continuously form and disband in the membrane (FIG. 14e). Pores conduct both water and ions (FIG. 14d), and leakage is dominated by larger more stable pores consisting of 10-12 peptides that form large aqueous channels lined with polar and charged side chains (FIG. 14c).

SEQUENCE DESCRIPTIONS

| SEQ ID | Name | Sequence |
|---|---|---|
| SEQ ID NO: 1 | DEE | GLLDLLELLLEAAG |
| SEQ ID NO: 2 | EEE | GLLELLELLLEAAG |
| SEQ ID NO: 3 | HEE | GLLHLLELLLEAAG |
| SEQ ID NO: 4 | KEE | GLLKLLELLLEAAG |
| SEQ ID NO: 5 | DHE | GLLDLLHLLLEAAG |
| SEQ ID NO: 6 | EHE | GLLELLHLLLEAAG |
| SEQ ID NO: 7 | HHE | GLLHLLHLLLEAAG |
| SEQ ID NO: 8 | KHE | GLLKLLHLLLEAAG |
| SEQ ID NO: 9 | DKE | GLLDLLKLLLEAAG |
| SEQ ID NO: 10 | EKE | GLLELLKLLLEAAG |
| SEQ ID NO: 11 | HKE | GLLHLLKLLLEAAG |
| SEQ ID NO: 12 | KKE | GLLKLLKLLLEAAG |
| SEQ ID NO: 13 | DEH | GLLDLLELLLHAAG |
| SEQ ID NO: 14 | EEH | GLLELLELLLHAAG |
| SEQ ID NO: 15 | HEH | GLLHLLELLLHAAG |
| SEQ ID NO: 16 | KEH | GLLKLLELLLHAAG |
| SEQ ID NO: 17 | DHH | GLLDLLHLLLHAAG |
| SEQ ID NO: 18 | EHH | GLLELLHLLLHAAG |
| SEQ ID NO: 19 | HHH | GLLHLLHLLLHAAG |
| SEQ ID NO: 20 | KHH | GLLKLLHLLLHAAG |
| SEQ ID NO: 21 | DKH | GLLDLLKLLLHAAG |
| SEQ ID NO: 22 | EKH | GLLELLKLLLHAAG |
| SEQ ID NO: 23 | HKH | GLLHLLKLLLHAAG |
| SEQ ID NO: 24 | KKH | GLLKLLKLLLHAAG |
| SEQ ID NO: 25 | DEK | GLLDLLELLLKAAG |
| SEQ ID NO: 26 | EEK | GLLELLELLLKAAG |
| SEQ ID NO: 27 | HEK | GLLHLLELLLKAAG |
| SEQ ID NO: 28 | KEK | GLLKLLELLLKAAG |
| SEQ ID NO: 29 | DHK | GLLDLLHLLLKAAG |
| SEQ ID NO: 30 | EHK | GLLELLHLLLKAAG |
| SEQ ID NO: 31 | HHK | GLLHLLHLLLKAAG |
| SEQ ID NO: 32 | KHK | GLLKLLHLLLKAAG |
| SEQ ID NO: 33 | DKK | GLLDLLKLLLKAAG |
| SEQ ID NO: 34 | EKK | GLLELLKLLLKAAG |
| SEQ ID NO: 35 | HKK | GLLHLLKLLLKAAG |
| SEQ ID NO: 36 | KKK | GLLKLLKLLLKAAG |
| SEQ ID NO: 37 | 25B2 | GLDDLAKLLLKLAG |

### REFERENCES

All patent and literature references cited in the present specification are hereby incorporated by reference in their entirety.
1. Nguyen, L. V.; Vanner, R.; Dirks, P.; Eaves, C. J., Cancer stem cells: an evolving concept. Nat Rev Cancer 2012, 12 (2), 133-43.
2. Plaks, V.; Kong, N.; Werb, Z., The cancer stem cell niche: how essential is the niche in regulating sternness of tumor cells? Cell Stem Cell 2015, 16 (3), 225-38.
3. Dean, M.; Fojo, T.; Bates, S., Tumour stem cells and drug resistance. Nat Rev Cancer 2005, 5 (4), 275-84.
4. Marx, J., Molecular biology. Cancer's perpetual source? Science 2007, 317 (5841), 1029-31.
5. Kaiser, J., The cancer stem cell gamble. Science 2015, 347 (6219), 226-9.
6. Pattabiraman, D. R.; Weinberg, R. A., Tackling the cancer stem cells—what challenges do they pose? Nat Rev Drug Discov 2014, 13 (7), 497-512.
7. Gupta, P. B.; Onder, T. T.; Jiang, G.; Tao, K.; Kuperwasser, C.; Weinberg, R. A.; Lander, E. S., Identification of selective inhibitors of cancer stem cells by high-throughput screening. Cell 2009, 138 (4), 645-659.
8. Bao, S.; Wu, Q.; McLendon, R. E.; Hao, Y.; Shi, Q.; Hjelmeland, A. B.; Dewhirst, M. W.; Bigner, D. D.; Rich, J. N., Glioma stem cells promote radioresistance by preferential activation of the DNA damage response. Nature 2006, 444 (7120), 756-60.
9. Papaccio, F.; Paino, F.; Regad, T.; Papaccio, G.; Desiderio, V.; Tirino, V., Concise Review: Cancer Cells, Cancer Stem Cells, and Mesenchymal Stem Cells: Influence in Cancer Development. Stem Cells Transl Med 2017, 6 (12), 2115-2125.
10. Izzedine, H.; Perazella, M. A., Anticancer Drug-Induced Acute Kidney Injury. Kidney Int Rep 2017, 2 (4), 504-514.
11. Rosner, M. H.; Perazella, M. A., Acute Kidney Injury in Patients with Cancer. N Engl J Med 2017, 377 (5), 500-501.
12. Rosner, M. H.; Capasso, G.; Perazella, M. A., Acute kidney injury and electrolyte disorders in the critically ill patient with cancer. Curr Opin Crit Care 2017, 23 (6), 475-483.
13. Wimley, W. C.; Hristova, K., Antimicrobial peptides: successes, challenges and unanswered questions. J Membr Biol 2011, 239 (1-2), 27-34.
14. Shai, Y., Mode of action of membrane active antimicrobial peptides. Biopolymers 2002, 66 (4), 236-48.
15. Gaspar, D.; Veiga, A. S.; Castanho, M. A., From antimicrobial to anticancer peptides. A review. Front Microbiol 2013, 4, 294.
16. Andreev, O. A.; Engelman, D. M.; Reshetnyak, Y. K., pH-sensitive membrane peptides (pHLIPs) as a novel class of delivery agents. Mol Membr Biol 2010, 27 (7), 341-52.
17. Andreev, O. A.; Karabadzhak, A. G.; Weerakkody, D.; Andreev, G. O.; Engelman, D. M.; Reshetnyak, Y. K., pH (low) insertion peptide (pHLIP) inserts across a lipid bilayer as a helix and exits by a different path. Proc Nat Acad Sci USA 2010, 107 (9), 4081-6.
18. An, M.; Wijesinghe, D.; Andreev, O. A.; Reshetnyak, Y. K.; Engelman, D. M., pH-(low)-insertion-peptide (pHLIP) translocation of membrane impermeable phalloidin toxin inhibits cancer cell proliferation. Proc Nat Acad Sci USA 2010, 107 (47), 20246-50.
19. Wyatt, L. C.; Moshnikova, A.; Crawford, T.; Engelman, D. M.; Andreev, O. A.; Reshetnyak, Y. K., Peptides of pHLIP family for targeted intracellular and extracellular delivery of cargo molecules to tumors. Proc Natl Acad Sci USA 2018, 115 (12), E2811-E2818.
20. Ishikawa, K.; Medina, S. H.; Schneider, J. P.; Klar, A. J. S., Glycan Alteration Imparts Cellular Resistance to a Membrane-Lytic Anticancer Peptide. Cell Chem Biol 2017, 24 (2), 149-158.
21. Freire, J. M.; Gaspar, D.; Veiga, A. S.; Castanho, M. A., Shifting gear in antimicrobial and anticancer peptides biophysical studies: from vesicles to cells. J Pept Sci 2015, 21 (3), 178-85.

1. A method for treating a cancer in a subject, comprising administering to the subject a pharmaceutical composition comprising an effective amount of one or more peptides having a sequence comprising the motif
GLLxLLxLLLxAAG, wherein each x is independently selected from arginine (R), histidine (H), lysine (K), aspartic acid (D) or glutamic acid (E), and one or more pharmaceutically acceptable excipients, thereby treating the cancer in the subject.

2. (canceled)

3. The method according to claim 1, wherein the motif is GLLxLLELLLxAAG.

4. The method according to claim 1, wherein the sequence does not comprise SEQ ID NO: 29 or SEQ ID NO: 33.

5. The method according to claim 1, wherein the pharmaceutical composition comprises one or more peptides comprising a sequence selected from SEQ ID NO: 2, SEQ ID NO: 4, SEQ ID NO: 6, SEQ ID NO: 14, SEQ ID NO: 25, SEQ ID NO: 26 or a mixture thereof.

6. The method according to claim 3, wherein the pharmaceutical composition comprises one or more peptides comprising a sequence selected from SEQ ID NO: 2, SEQ ID NO: 4, SEQ ID NO: 14, SEQ ID NO: 25, SEQ ID NO: 26 or a mixture thereof.

7. The method according to claim 3, wherein the sequence comprises SEQ ID NO: 25 and/or SEQ ID NO: 26.

8. The method according to claim 1, wherein the cancer is breast cancer.

9. The method according to claim 1, wherein the motif further comprises a tryptophan residue (W) at the C terminus.

10. The method according to claim 1, wherein the peptide sequence consists of the motif GLLxLLxLLLxAAG.

11. The method according to claim 1, further comprising administering an effective amount of a chemotherapy agent to the subject.

12. The method according to claim 1, comprising administering the pharmaceutical composition intravenously.

13. The method according to claim 1, wherein the effective amount is a dosage ranging from 1 nM to about 10,000 nM.

14. The method according to claim 1, wherein the one or more peptides is in the L form.

15. The method according to claim 1, wherein the peptide forms an alpha helical assembly.

16. The method according to claim 1, wherein the peptide forms a pore in a cancer cell membrane.

17. (canceled)

18. A peptide having a sequence comprising the motif GLLxLLELLLxAAG, wherein each x is independently selected from arginine (R), histidine (H), lysine (K), aspartic acid (D) or glutamic acid (E).

19. A peptide having a sequence comprising the motif GLLxLLxLLLxAAG, wherein each x is independently selected from arginine (R), histidine (H), lysine (K), aspartic acid (D) or glutamic acid (E) and wherein the sequence does not comprise SEQ ID NO: 29 or SEQ ID NO: 33.

20. The peptide according to claim 19, wherein the sequence comprises SEQ ID NO: 2, SEQ ID NO: 4, SEQ ID NO: 14, SEQ ID NO: 25, SEQ ID NO: 26 or a mixture thereof.

21. The peptide according to claim 18, wherein the sequence comprises SEQ ID NO: 2, SEQ ID NO: 4, SEQ ID NO: 14, SEQ ID NO: 25, SEQ ID NO: 26 or a mixture thereof.

22. The peptide according to claim 21, wherein the sequence comprises SEQ ID NO: 25 and/or SEQ ID NO: 26.

23. The peptide according to claim 18, wherein the motif further comprises a tryptophan residue (W) at the C terminus.

24. The peptide according to claim 18, wherein the peptide sequence consists of the motif GLLxLLxLLLxAAG.

25. The peptide according to claim 18, wherein the peptide is in the L form.

26. The peptide according to claim 18, wherein the peptide forms an alpha helical assembly.

27. The peptide according to claim 18, wherein the peptide forms a pore in a cancer cell membrane.

28. A kit for treating or preventing cancer comprising a pharmaceutically acceptable composition comprising the peptide of claim 18 and one or more pharmaceutically acceptable excipients.

29. A kit according to claim 28, wherein the cancer is breast cancer.

30. A kit according to claim 28, further comprising a chemotherapeutic agent.

31. A nucleotide sequence encoding a peptide comprising the sequence of any one of SEQ ID NO: 1 to 36.

32. A vector comprising the nucleic acid sequence of claim 31.

33. A pharmaceutically acceptable composition comprising one or more peptides according to claim 18, and one or more pharmaceutically acceptable excipients.


SEQUENCE LISTING (39 sequences)

SEQ ID NO: 1 - protein, 14 aa, Artificial Sequence
  Laboratory synthesized peptide DEE
GLLDLLELLLEAAG

SEQ ID NO: 2 - protein, 14 aa, Artificial Sequence
  Laboratory synthesized peptide EEE
GLLELLELLLEAAG

SEQ ID NO: 3 - protein, 14 aa, Artificial Sequence
  Laboratory synthesized peptide HEE
GLLHLLELLLEAAG

SEQ ID NO: 4 - protein, 14 aa, Artificial Sequence
  Laboratory synthesized peptide KEE
GLLKLLELLLEAAG

SEQ ID NO: 5 - protein, 14 aa, Artificial Sequence
  Laboratory synthesized peptide DHE
GLLDLLHLLLEAAG

SEQ ID NO: 6 - protein, 14 aa, Artificial Sequence
  Laboratory synthesized peptide EHE
GLLELLHLLLEAAG

SEQ ID NO: 7 - protein, 14 aa, Artificial Sequence
  Laboratory synthesized peptide HHE
GLLHLLHLLLEAAG

SEQ ID NO: 8 - protein, 14 aa, Artificial Sequence
  Laboratory synthesized peptide KHE
GLLKLLHLLLEAAG

SEQ ID NO: 9 - protein, 14 aa, Artificial Sequence
  Laboratory synthesized peptide DKE
GLLDLLKLLLEAAG

SEQ ID NO: 10 - protein, 14 aa, Artificial Sequence
  Laboratory synthesized peptide EKE
GLLELLKLLLEAAG

SEQ ID NO: 11 - protein, 14 aa, Artificial Sequence
  Laboratory synthesized peptide HKE
GLLHLLKLLLEAAG

SEQ ID NO: 12 - protein, 14 aa, Artificial Sequence
  Laboratory synthesized peptide KKE
GLLKLLKLLLEAAG

SEQ ID NO: 13 - protein, 14 aa, Artificial Sequence
  Laboratory synthesized peptide DEH
GLLDLLELLLHAAG

SEQ ID NO: 14 - protein, 14 aa, Artificial Sequence
  Laboratory synthesized peptide EEH
GLLELLELLLHAAG

SEQ ID NO: 15 - protein, 14 aa, Artificial Sequence
  Laboratory synthesized peptide HEH
GLLHLLELLLHAAG

SEQ ID NO: 16 - protein, 14 aa, Artificial Sequence
  Laboratory synthesized peptide KEH
GLLKLLELLLHAAG

SEQ ID NO: 17 - protein, 14 aa, Artificial Sequence
  Laboratory synthesized peptide DHH
GLLDLLHLLLHAAG

SEQ ID NO: 18 - protein, 14 aa, Artificial Sequence
  Laboratory synthesized peptide EHH
GLLELLHLLLHAAG

SEQ ID NO: 19 - protein, 14 aa, Artificial Sequence
  Laboratory synthesized peptide HHH
GLLHLLHLLLHAAG

SEQ ID NO: 20 - protein, 14 aa, Artificial Sequence
  Laboratory synthesized peptide KHH
GLLKLLHLLLHAAG

SEQ ID NO: 21 - protein, 14 aa, Artificial Sequence
  Laboratory synthesized peptide DKH
GLLDLLKLLLHAAG

SEQ ID NO: 22 - protein, 14 aa, Artificial Sequence
  Laboratory synthesized peptide EKH
GLLELLKLLLHAAG

SEQ ID NO: 23 - protein, 14 aa, Artificial Sequence
  Laboratory synthesized peptide HKH
GLLHLLKLLLHAAG

SEQ ID NO: 24 - protein, 14 aa, Artificial Sequence
  Laboratory synthesized peptide KKH
GLLKLLKLLLHAAG

SEQ ID NO: 25 - protein, 14 aa, Artificial Sequence
  Laboratory synthesized peptide DEK
GLLDLLELLLKAAG

SEQ ID NO: 26 - protein, 14 aa, Artificial Sequence
  Laboratory synthesized peptide EEK
GLLELLELLLKAAG

SEQ ID NO: 27 - protein, 14 aa, Artificial Sequence
  Laboratory synthesized peptide HEK
GLLHLLELLLKAAG

SEQ ID NO: 28 - protein, 14 aa, Artificial Sequence
  Laboratory synthesized peptide KEK
GLLKLLELLLKAAG

SEQ ID NO: 29 - protein, 14 aa, Artificial Sequence
  Laboratory synthesized peptide DHK
GLLDLLHLLLKAAG

SEQ ID NO: 30 - protein, 14 aa, Artificial Sequence
  Laboratory synthesized peptide EHK
GLLELLHLLLKAAG

SEQ ID NO: 31 - protein, 14 aa, Artificial Sequence
  Laboratory synthesized peptide HHK
GLLHLLHLLLKAAG

SEQ ID NO: 32 - protein, 14 aa, Artificial Sequence
  Laboratory synthesized peptide KHK
GLLKLLHLLLKAAG

SEQ ID NO: 33 - protein, 14 aa, Artificial Sequence
  Laboratory synthesized peptide DKK
GLLDLLKLLLKAAG

SEQ ID NO: 34 - protein, 14 aa, Artificial Sequence
  Laboratory synthesized peptide EKK
GLLELLKLLLKAAG

SEQ ID NO: 35 - protein, 14 aa, Artificial Sequence
  Laboratory synthesized peptide HKK
GLLHLLKLLLKAAG

SEQ ID NO: 36 - protein, 14 aa, Artificial Sequence
  Laboratory synthesized peptide KKK
GLLKLLKLLLKAAG

SEQ ID NO: 37 - protein, 14 aa, Artificial Sequence
  Laboratory synthesized peptide 25B2
GLDDLAKLLLKLAG

SEQ ID NO: 38 - protein, 14 aa, Artificial Sequence
  Laboratory synthesized peptide GLLxLLxLLLxAAG
  MISC_FEATURE (4)..(4): Xaa is selected from arginine (R), histidine (H), lysine (K), aspartic acid (D) or glutamic acid (E)
  MISC_FEATURE (7)..(7): Xaa is selected from arginine (R), histidine (H), lysine (K), aspartic acid (D) or glutamic acid (E)
  MISC_FEATURE (11)..(11): Xaa is selected from arginine (R), histidine (H), lysine (K), aspartic acid (D) or glutamic acid (E)
GLLXLLXLLLXAAG

SEQ ID NO: 39 - protein, 14 aa, Artificial Sequence
  Laboratory synthesized peptide GLLxLLELLLxAAG
  MISC_FEATURE (4)..(4): Xaa is selected from arginine (R), histidine (H), lysine (K), aspartic acid (D) or glutamic acid (E)
  MISC_FEATURE (11)..(11): Xaa is selected from arginine (R), histidine (H), lysine (K), aspartic acid (D) or glutamic acid (E)
GLLXLLELLLXAAG